(19) United States
(12) Patent Application Publication
(10) Pub. No.: US 2021/0107886 A1
(43) Pub. Date: Apr. 15, 2021

(54) PREPARATION OF CYCLIC ACETALS FOR FUEL

(71) Applicant: TRIAD NATIONAL SECURITY, LLC, LOS ALAMOS, NM (US)

(72) Inventors: ANDREW SUTTON, LOS ALAMOS, NM (US); CAMERON M. MOORE, LOS ALAMOS, NM (US); ORION STAPLES, PHILADELPHIA, PA (US)

(21) Appl. No.: 17/033,142

(22) Filed: Sep. 25, 2020

Related U.S. Application Data
(60) Provisional application No. 62/905,731, filed on Sep. 25, 2019.

(51) Int. Cl. C07D 317/12; C10L 1/185; C10L 10/12; C10L 10/14; C10L 10/06

(52) U.S. Cl. CPC C07D 317/12; C10L 1/1855; C10L 10/12; C10L 2270/026; C10L 10/06; C10L 2200/0476; C10L 10/14

(57) ABSTRACT

The present disclosure relates to methods for preparing cyclic acetals from the catalytic transformation of bio-derived aldehydes with 2,3-butanediol. The methods operate in a solvent-free reaction system and provide isolation of the product acetals through phase separation and subsequent decantation. The cyclic acetals exhibit excellent properties for blending with diesel fuel, including increased cetane number and density, and decreased viscosity, freezing point, and soot formation.

### CROSS-REFERENCE TO RELATED APPLICATIONS

This application claims the benefit of priority to U.S. Provisional Patent Application No. 62/905,731, filed on Sep. 25, 2019, the content of which is incorporated by reference herein in its entirety for all purposes.

### GOVERNMENT INTEREST

The United States government has certain rights in this invention pursuant to Contract No. 89233218CNA000001 between the United States Department of Energy and TRIAD National Security, LLC for the operation of Los Alamos National Laboratory.

### FIELD

The subject matter described herein relates to the preparation of cyclic acetals from bio-derived aldehydes for renewable diesel fuels.

### BACKGROUND

Significant efforts have been focused on developing new methods to convert non-food biomass feedstocks' into suitable liquid fuels that meet or exceed the performance and physical properties of gasoline, diesel and/or jet fuels.² This approach aims to supplant single use carbon with renewable carbon that can quickly be reincorporated into the carbon cycle. Biodiesel has been employed³ since the advent of the diesel engine and traditionally refers to the transesterification products of animal fats, vegetable oils, or recycled restaurant grease with methanol to produce fatty acid methyl ester (FAME) fuels and glycerol.⁴ These biodiesels have some favorable properties when compared to traditional diesel including significant reduction in aromatic emissions, increased lubricity, and increased cetane number (a measure of the ignitability of a fuel in a compression ignition engine).⁴⁻⁶ Another aspect of these biofuels is the presence of oxygen-containing functional groups, in contrast to petroleum diesel, which contains <0.6 wt. % oxygen.^{5,7} This offers a significant benefit for current biofuels over petroleum diesel, as the oxygen helps increase combustion efficiency, reduce NOx emissions, and produce significantly less particulate matter.⁵⁻⁷ While these properties encourage further development of oxygenated biodiesel, there are several drawbacks that limit their extensive application. These drawbacks include a much higher kinematic viscosity than petroleum diesel and a high freezing point, both of which render these fuels less likely to be suitable for cold weather applications.⁸ Therefore, there is a need for the preparation of advanced renewable diesel fuels that exhibit improved performance over traditional biodiesel. The subject matter described herein addresses this problem.

### BRIEF SUMMARY

In certain aspects, the subject matter described herein is directed to a process for the preparation of a diesel fuel additive compound of Formula I,

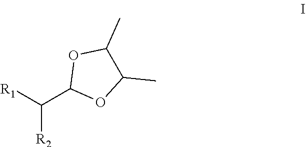

wherein R₁ is C₁₋₈ alkyl or C₂₋₆ alkyl and R₂ is selected from the group consisting of H, C₁₋₈ alkyl, and C₂₋₆ alkyl,

the process comprising:

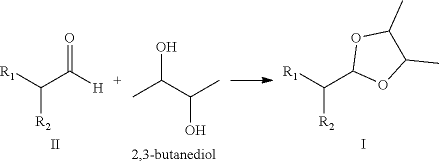

contacting a compound of formula II with 2,3-butanediol in the presence of a catalyst to form a solution;

heating said solution at a temperature of about 40° C.; and

allowing said solution to separate into an upper phase and a lower phase.
In other aspects, the subject matter described herein is directed to a method of improving a property of a diesel fuel, comprising:

contacting a diesel fuel with a compound of Formula I,

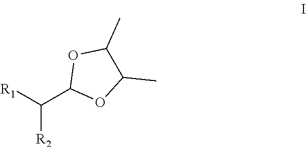

wherein R₁ is C₁₋₈ alkyl or C₂₋₆ alkyl and R₂ is selected from the group consisting of H, C₁₋₈ alkyl, and C₂₋₆ alkyl,
wherein said property is selected from the group consisting of cetane number, freezing point, viscosity, yield sooting index, net heat of combustion, cloud point, and density.
   In other aspects, the subject matter described herein is directed to a diesel fuel composition comprising a diesel fuel and a compound of Formula I,

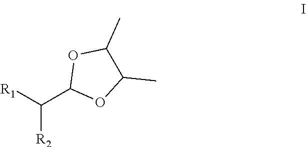

wherein R₁ is C₁₋₈ alkyl or C₂₋₆ alkyl and R₂ is selected from the group consisting of H, C₁₋₈ alkyl, and C₂₋₆ alkyl.

These and other aspects are described fully herein.

### BRIEF DESCRIPTION OF THE FIGURES

FIG. 1A is a chemical scheme demonstrating the acetalization of bio-derived aldehydes with 2,3-butanediol to yield substituted 1,3-dioxolanes.

FIG. 1B shows a vial containing 2-ethylbutyraldehyde and BDO. Amberlyst 15™ was added to the vial in the absence of any solvent. The two compounds were miscible when mixed.

FIG. 1C shows a distinct phase separation after stirring a vial containing 2-ethylbutyraldehyde, BDO, and Amberlyst 15 for 5 h under mild (≤40° C.) temperatures. The upper organic phase contained only the desired 1,3-dioxolane, while the unreacted BDO and the produced water formed a bottom, aqueous layer.

FIG. 2 shows a 400 MHz ¹H NMR spectrum of (1). The inset images show magnified portions of the NMR spectrum.

FIG. 3 shows a 101 MHz ¹³C NMR spectrum of (1). The inset images show magnified portions of the NMR spectrum.

FIG. 4 shows a gas chromatogram trace of (1).

FIG. 5 shows a mass spectrum of (1).

FIG. 6 shows a DSC trace of (1).

FIG. 7 shows a 400 MHz ¹H NMR spectrum of (2). The inset images show magnified portions of the NMR spectrum.

FIG. 8 shows a 101 MHz ¹³C NMR spectrum of (2). The inset images show magnified portions of the NMR spectrum.

FIG. 9 shows a gas chromatogram trace of (2).

FIG. 10 shows a mass spectrum of (2).

FIG. 11 shows a differential scanning calorimetry trace of (2).

FIG. 12 shows a 400 MHz ¹H NMR spectrum of (3). The inset images show magnified portions of the NMR spectrum.

FIG. 13 shows a 101 MHz ¹³C NMR spectrum of (3). The inset images show magnified portions of the NMR spectrum.

FIG. 14 shows a gas chromatogram trace of (3).

FIG. 15 shows a mass spectrum of (3).

FIG. 16 shows a differential scanning calorimetry trace of (3).

FIG. 17 shows a 400 MHz ¹H NMR spectrum of (4). The inset images show magnified portions of the NMR spectrum.

FIG. 18 shows a 101 MHz ¹³C NMR spectrum of (4). The inset images show magnified portions of the NMR spectrum.

FIG. 19 shows a gas chromatogram trace of (4).

FIG. 20 shows a mass spectrum of (4).

FIG. 21 shows a differential scanning calorimetry trace of (4).

FIG. 22 shows a 400 MHz ¹H NMR spectrum of (5). The inset images show magnified portions of the NMR spectrum.

FIG. 23 shows a 101 MHz ¹³C NMR spectrum of (5). The inset images show magnified portions of the NMR spectrum.

FIG. 24 shows a gas chromatogram mass spectrum trace of (5).

FIG. 25 shows a mass spectrum of (5).

FIG. 26 shows a differential scanning calorimetry trace of (5).

FIG. 27 shows gas chromatography mass spectrometry traces of catalyst recycle runs 1-10.

FIG. 28A shows a representative reaction for catalyst recycling.

FIG. 28B shows the isolated yields and conversions for each of the recycling runs. *Indicates when the aqueous phase was removed to provide adequate space for additional reactants.

FIG. 29 shows an NMR spectrum of (3) generated using Nafion as a catalyst.

FIG. 30 shows a plot indicating how cetane number is influenced by different dioxolane blends.

FIG. 31A shows a plot of the percent volume change of fluoroelastomers as a function of the mass concentration of dioxolane (1).

FIG. 31B shows a plot of the percent volume change of fluoroelastomers as a function of the mass concentration of dioxolane (2).

FIG. 32A shows a plot of the percent volume change of neoprene, silicone, SBR, and EPDM as a function of the mass concentration of dioxolane (1).

FIG. 32B shows a plot of the percent volume change of neoprene, silicone, SBR, and EPDM as a function of the mass concentration of dioxolane (2).

FIG. 33A shows a plot of the percent volume change of polyurethane, ECO, OZO, and HNBR as a function of the mass concentration of dioxolane (1).

FIG. 33B shows a plot of the percent volume change of polyurethane, ECO, OZO, and HNBR as a function of the mass concentration of dioxolane (2).

FIG. 34A shows a plot of the percent volume change of nitrile rubbers as a function of the mass concentration of dioxolane (1).

FIG. 34B shows a plot of the percent volume change of nitrile rubbers as a function of the mass concentration of dioxolane (2).

### DETAILED DESCRIPTION

Described herein are efficient methods for the catalytic transformation of bio-derived aldehydes with BDO to afford substituted 1,3-dioxolanes with high yield and purity. The methods disclosed herein operate at mild temperatures, use readily available reagents, and provide easy isolation of the product through phase separation and subsequent decantation in a solvent-free reaction system. The 1,3-dioxolanes exhibit excellent properties for blending with diesel fuel, including increased cetane number and density, and decreased viscosity, freezing point, and soot formation.

Ethanol has gained interest as a bio-derived building block for fuel production. This interest is largely due to the high volume and growth of global ethanol production (2.65×10¹⁰ U.S. gal in 2016) versus its limited applicability in road transport fuels by the current “blend wall,” which imposes limits (typically 10-15%) on the volume that is allowed to be blended with gasoline.⁹ A method to produce C₄₋₈ aldehydes from the condensation of acetaldehyde, the dehydrogenation product of ethanol, may be applied in the manufacture of fuels.¹⁰ While several routes are available to produce aldehydes, including the hydroformylation of propene to generate butyraldehyde¹¹ or the oxidation of alcohols,¹² these routes typically rely on hydrocarbons derived from petroleum (i.e. propene). Other efforts have focused on the production of hydrocarbons directly from ethanol,¹³ but these products require additional upgrading (akin to petroleum)¹⁴ to introduce additional beneficial functional groups. By contrast, in order to capitalize on the abundant functional groups present in biomass, bioderived precursors are implemented to provide access to industrially-relevant aldehydes (i.e. butyraldehyde and 2-ethylhexanal).¹⁰ Further stepwise and selective derivatization of these aldehydes (i.e. hydrogenation or acetalization) provides access to both relevant chemical precursors and drop-in renewable fuels.

The aldehyde moiety provides a functional handle through which various reaction pathways could modify the end product to suit a range of fuel applications. A traditional approach involves performing hydrodeoxygenation (HDO) of the bio-derived aldehydes to obtain the respective alkanes. However, these hydrocarbons do not have octane or cetane numbers high enough for gasoline or diesel use, respectively, and would decrease the overall atom efficiency of the process.¹⁵ To this end, as disclosed herein, several chemical additives were prepared in an effort to increase the cetane number of diesel fuels and to decrease fuel viscosity, thereby enhancing combustion efficiency and fuel economy.¹⁶⁻¹⁷ Consequently, the subject matter disclosed herein is directed to the incorporation of additional carbon into aldehydes via acid catalysed acetalization to obtain fuels that both retain valuable oxygen as well as increased renewable carbon. Rather than extending the carbon chain and subsequently removing all the oxygen via HDO, efforts were focused on the preparation of oxygenated hydrocarbons, similar to traditional biodiesel, which burns cleaner than petroleum diesel.^{18,19} Several advantages of ethers over fatty acids exist; namely, the ethers are less viscous, less likely to freeze, and are more resistant to common microbes in the environment.^{16,20}

It has been demonstrated that bio-derived aldehydes can be readily transformed into diethyl acetals via acid catalyzed acetalization with ethanol.¹⁰ The subject matter described herein, however, is directed to the preparation of 1,3-dioxolane compounds as renewable diesel molecules, as they may exhibit higher stability than the corresponding diethyl acetals. Through careful selection of the vicinal diol, it would be possible to incorporate additional renewable carbon into the molecule, while maintaining the oxygen atoms. For example, ethylene glycol (EG) and 2,3-butanediol (BDO) are both vicinal diols; however, BDO would incorporate twice as much renewable carbon, which is available via fermentation of biomass with Zymomonas mobilis.²¹

It has been demonstrated that certain dioxolanes can be produced from BDO via a dehydration/acetalization reaction, as well as from the condensation of bio-derived methylketones with diols (such as BDO, 1,2-propanediol and ethylene glycol).^{18,22} While the dioxolane mixture obtained (2-ethyl-2,4,5-trimethyl-1,3-dioxolane and isomers) exhibited good fuel properties for use in spark ignition engines (anti-knock index=90.1) and could be blended with gasoline without introducing adverse water solubility, the fuel exhibited a poor derived cetane number (DCN) of 22.9.²² Dioxolane fuels more suitable for compression ignition engines were produced from the condensation of methyl ketones with diols (obtaining three dioxolanes 2,4,5-trimethyl-2-undecyl-1,3-dioxolane (H1), 2,4-dimethyl-2-undecyl-1,3-dioxolane (H2), and 2-methyl-2-undecyl-1,3-dioxolane (H3)).¹⁸ These fuels showed cetane numbers and viscosities similar to that of biodiesel. While the before-mentioned reports demonstrate the potential for dioxolanes as renewable fuel blendstocks, the exploration of alternative sources of renewable carbon and methods for their production could further reveal the impact of these potential biofuels.

The methods described herein for the formation of 1,3-dioxolanes could be used for the simultaneous production and extraction of renewable diesel additives directly from BDO fermentation broths using bio-derived aldehydes.²⁹ As such, the methods described herein provide an opportunity to further diversify the use of ethanol as a renewable diesel fuel and provide performance advantages to the consumer.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

As used herein, “alkyl” refers to an unbranched or branched saturated hydrocarbon chain. As used herein, alkyl has 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), 1 to 5 carbon atoms (i.e., C₁₋₅ alkyl), 2 to 6 carbon atoms (i.e., C₂₋₆ alkyl), or 3 to 5 carbon atoms (i.e., C₃₋₅ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

As used herein, “BDO” refers to 2,3-butanediol.

As used herein, the term “contacting” refers to allowing two or more reagents to contact each other. The contact may or may not be facilitated by mixing, agitating, stirring, and the like.

As used herein, “diesel fuel additive” refers to a compound that is added to a diesel fuel. In certain embodiments, the diesel fuel additive possesses one or more properties, such as cetane number, freezing point, viscosity, yield sooting index, net heat of combustion, cloud point, or density, which improves the same property of the diesel fuel.

As used herein, “biodiesel fuel” refers to a fuel developed from vegetable oil or animal fat.

As used herein, “petroleum diesel fuel” refers to a fuel produced by distilling crude oil.

As used herein, “ULSD” refers to ultra-low-sulfur diesel.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the presently described subject matter includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In some embodiments, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Additional definitions may be provided herein.

### II. Synthetic Methods

In an aspect, the subject matter described herein is directed to processes for the preparation of a diesel fuel additive compound of Formula I,

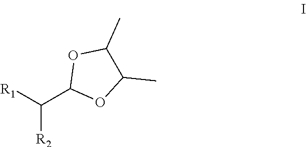

wherein R₁ is C₁₋₈ alkyl or C₂₋₆ alkyl and R₂ is selected from the group consisting of H, C₁₋₈ alkyl, and C₂₋₆ alkyl,

the process comprising:

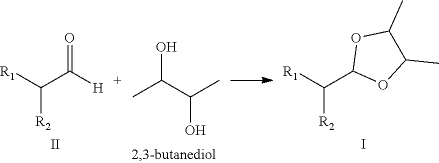

contacting a compound of formula II with 2,3-butanediol in the presence of a catalyst to form a solution;

heating said solution at a temperature of about 40° C.; and

allowing said solution to separate into an upper phase and a lower phase.

In certain embodiments, R¹ is C₂ alkyl and R² is C₂ alkyl. In certain embodiments, R¹ is C₄ alkyl and R² is C₂ alkyl. In certain embodiments, R¹ is C₂ alkyl and R² is H. In certain embodiments, R¹ is C₄ alkyl and R² is H. In certain embodiments, R¹ is C₆ alkyl and R² is H.

In certain embodiments, the diesel fuel additive compound of Formula I is selected from the group consisting of

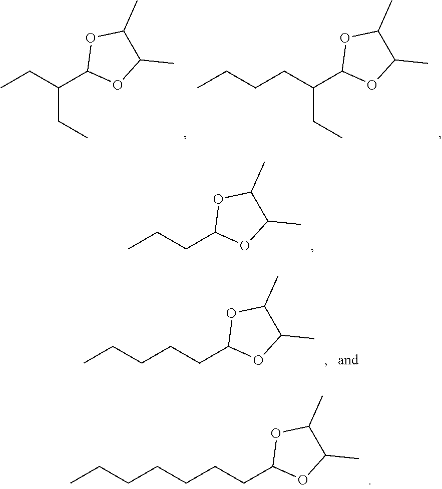

In certain embodiments, the catalyst is a solid acid catalyst selected from a zeolite-based solid acid, a polymer-based solid acid, a carbon-based solid acid, a hydroxyapatite-based solid acid, a zirconia-based solid acid, or a silica-based solid acid. In certain embodiments, the solid acid catalyst can be a sulfonated tetrafluoroethylene-based fluoropolymer-copolymer (e.g., a NAFION® catalyst), a styrene-divinylbenzene polymer acid (e.g., AMBERLYST® catalyst, such as an AMBERLYST® 15 ion exchange resin, an AMBERLYST® 36 ion exchange resin, or the like), a zeolite-based polymer 50 (e.g., H-ZSM-5 or the like), or a combination thereof. In certain embodiments, the catalyst is Amberlyst-15. In certain embodiments, the catalyst is NAFION.

In certain embodiments, the solution is heated at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.

In certain embodiments, the process further comprises separating the upper phase from the solution, wherein the upper phase comprises the compound of Formula I.

In certain embodiments, the process further comprises repeating the contacting, wherein the 2,3-butanediol is unreacted 2,3-butanediol from the lower phase. In certain embodiments, the contacting is repeated 1, 2, 3, 4, 5, 6, 7, 8, 9, 10, 11, 12, 13, 14, 15, 16, 17, 18, 19, 20, 21, 22, 23, 24, or 25 times.

In certain embodiments, the heating is carried out between about 30 minutes and about 48 hours. In certain embodiments, the heating is carried out between about 1 hour and 36 hours, about 2 hours and about 24 hours, about 1 hour and about 15 hours, about 2 hours and about 10 hours, about 3 hours and about 7 hours, about 4 hours and about 6 hours, or about 5 hours and about 9 hours.

In certain embodiments, the 2,3-butanediol is present in an amount ranging from about 1.25 to about 2.75 moles relative to 1 mole of the compound of Formula II. In certain embodiments, the 2,3-butanediol is present in an amount ranging from about 0.5 moles to about 6 moles, from about 0.5 moles to about 4 moles, to about 0.75 moles to about 5 moles, from about 1 mole to about 4.5 moles from about 1.25 moles to about 3.25 moles, from about 1.5 moles to about 3 moles, from about 1.75 moles to about 3.75 moles, from about 2 moles to about 2.25 moles, from about 2.25 moles to about 2.75 moles, or from about 1.75 moles to about 3 moles relative to 1 mole of the compound of Formula II.

In certain embodiments, the subject matter disclosed herein is directed to a method of improving a property of a diesel fuel, comprising:
contacting a diesel fuel with a compound of Formula I,

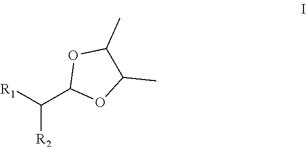

wherein R₁ is C₁₋₈ alkyl or C₂₋₆ alkyl and R₂ is selected from the group consisting of H, C₁₋₈ alkyl, and C₂₋₆ alkyl,
wherein the property is selected from the group consisting of cetane number, freezing point, viscosity, yield sooting index, net heat of combustion, density, and cloud point.

As described herein, the freezing point of the diesel fuel can be improved when the compound of Formula I being contacted with the diesel fuel has a freezing point that is lower than that of the diesel fuel.

As described herein, the viscosity of the diesel fuel can be improved when the compound of Formula I being contacted with the diesel fuel has a viscosity that is lower than that of the diesel fuel.

As described herein, the yield sooting index of the diesel fuel can be improved when the compound of Formula I being contacted with the diesel fuel has a yield sooting index that is lower than that of the diesel fuel.

As described herein, the yield sooting index of the diesel fuel can be improved when the compound of Formula I being contacted with the diesel fuel has a yield sooting index that is lower than that of the diesel fuel.

As described herein, the net heat of combustion of the diesel fuel can be improved when the compound of Formula I being contacted with the diesel fuel has a net heat of combustion that is lower than that of the diesel fuel.

As described herein, the viscosity of the diesel fuel can be improved when the compound of Formula I being contacted with the diesel fuel has a viscosity that does not decrease the density of the fuel below 0.82 g mL⁻¹ (See references 17 and 20).

In certain embodiments, the property of the diesel fuel being improved is viscosity and the compound of Formula 1 has a viscosity of about 0.9 mm² s⁻¹ to about 2.4 mm² s⁻¹ at 40° C. In certain embodiments, the compound of Formula 1 has a viscosity of about 0.5 mm² s⁻¹ to about 3 mm² s⁻¹, about 0.75 mm² s⁻¹ to about 2.5 mm² s⁻¹, or about 0.8 mm² s⁻¹ to about 2.75 mm² s⁻¹. In certain embodiments, the compound of Formula 1 has a viscosity of about 1.26 mm² s⁻¹, 1.88 mm² s⁻¹, 0.94 mm² s⁻¹, 1.49 mm² s⁻¹, or about 2.34 mm² s⁻¹ at 40° C.

In certain embodiments, the property of the diesel fuel being improved is cetane number and the compound of Formula 1 has a derived cetane number of about 45 to about 70. In certain embodiments, the compound of Formula 1 has a derived cetane number of about 40 to about 75, about 30 to about 70, or about 33 to about 75. In certain embodiments, the compound of Formula 1 has a derived cetane number of about 32, 33, 34, 35, 36, 37, 38, 39, 40, 41, 42, 43, 44, 45, 64, 65, 33, 34, 48, 49, 68, 69, or 70.

In certain embodiments, the property of the diesel fuel being improved is freezing point and the compound of Formula I has a freezing point of less than −100° C. In certain embodiments, the compound of Formula I has a freezing point of less than −105, −110, −115, −125, −130, −135, −140, −145, or −150.

In certain embodiments, the property of the diesel fuel being improved is density and the compound of Formula I has a density of about 0.883 g/mL to about 0.894 g/mL at 25° C. In certain embodiments, the compound of Formula I has a density of about 0.880 g/mL to about 0.895 g/mL at 25° C. In certain embodiments, the compound of Formula I has a density of about 0.893 g/mL, about 0.885 g/mL, about 0.894 g/mL, 0.887 g/mL, or about 0.883 g/mL at 25° C.

In certain embodiments, the property of the diesel fuel being improved is net heat of combustion and the compound of Formula I exhibits a net heat of combustion of about 27.67 MJ/L to about 30.41 MJ/L. In certain embodiments, the compound of Formula I exhibits a net heat of combustion of about 25 MJ/L to about 35 MJ/L or about 27.60 MJ/L to about 30.50 MJ/L. In certain embodiments, the compound of Formula I exhibits a net heat of combustion of about 29.45 MJ/L, 30.41 MJ/L, 27.67 MJ/L, 29.20 MJ/L, or about 30.06 MJ/L.

In certain embodiments, the property of the diesel fuel being improved is yield sooting index and the compound of Formula I exhibits a exhibits a yield sooting index of about 36 to about 69. In certain embodiments, the compound of Formula I exhibits a exhibits a yield sooting index of about 30 to about 75, about 35 to about 70, or about 40 to about 65. In certain embodiments, the compound of Formula I exhibits a exhibits a yield sooting index of about 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 69, or 70.

As described herein, the cetane number of the diesel fuel can be improved when the compound of Formula I being contacted with the diesel fuel has a cetane number that is higher than that of the diesel fuel. In certain embodiments, the property of the diesel fuel being improved is cetane number, and the compound of Formula I improves (increases) the cetane number of the diesel fuel by at least 3%. In certain embodiments, the property is cetane number, and the compound of Formula I improves the cetane number of the diesel fuel by at least 1% m 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 5100, 52%, 53%, 54%, 55%, 56%, 57%, 58%, 59%, 60%, 61%, 62%, 63%, 64%, 65%, 66%, 67%, 68%, 69%, or 70%.

As described herein, the cloud point of the diesel fuel can be improved when the compound of Formula I being contacted with the diesel fuel has a cloud point that is lower than that of the diesel fuel. In certain embodiments, the property of the diesel fuel being improved is cloud point, and the compound of Formula I improves (decreases) the cloud point of the diesel fuel by at least 2%. In certain embodiments, the property is cloud point, and the compound of Formula I improves the cloud point of the diesel fuel by at least 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%.

In certain embodiments, the diesel fuel is contacted with a compound of Formula I, in a ratio of about 1:10 (compound of formula I: diesel fuel). In certain embodiments, the diesel fuel is contacted with a compound of Formula I, in a ratio of about 2:10 (compound of formula I: diesel fuel). In certain embodiments, the diesel fuel is contacted with a compound of Formula I, in a ratio of about 3:10 (compound of formula I: diesel fuel).

In certain embodiments, the subject matter disclosed herein is directed to a method of improving a property of a diesel fuel, comprising contacting a diesel fuel with a compound of Formula I, wherein said compound is

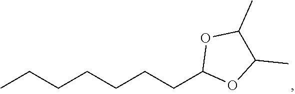

and wherein said compound of Formula I is contacted with said diesel fuel in a ratio of 3:10 (compound of formula I: diesel fuel), wherein said property is cetane number, and said compound of Formula I improves said cetane number of said diesel fuel by at least 10%, 11%, 12%, 13%, 14%, 15%, 16%, or 17%.

In certain embodiments, the subject matter disclosed herein is directed to a method of improving a property of a diesel fuel, comprising contacting a diesel fuel with a compound of Formula I, wherein said compound is

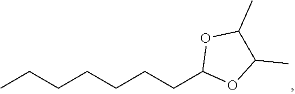

and wherein said compound of Formula I is contacted with said diesel fuel in a ratio of 3:10 (compound of formula I: diesel fuel), wherein said property is cetane number, and said compound of Formula I improves said cetane number of said diesel fuel by at least 7%, 8%, 9%, 10%, 11%, 12%, 13%, 14%, or 15%.

In certain embodiments, the subject matter disclosed herein is directed to a method of improving a property of a diesel fuel, comprising contacting a diesel fuel with a compound of Formula I, wherein said compound of Formula I is selected from the group consisting of

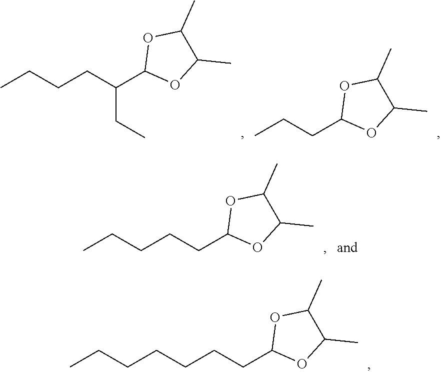

wherein said compound of Formula I is contacted with said diesel fuel in a ratio of 1:10, 2:10, or 3:10 (compound of formula I: diesel fuel), and wherein said property is cetane number, and said compound of Formula I improves said cetane number of said diesel fuel by at least 0.1%, 0.2%, 0.3%, 0.4%, 0.5%, 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%.

In certain embodiments, the subject matter disclosed herein is directed to a method of improving a property of a diesel fuel, comprising contacting a diesel fuel with a compound of Formula I, wherein said compound of Formula I is selected from the group consisting of

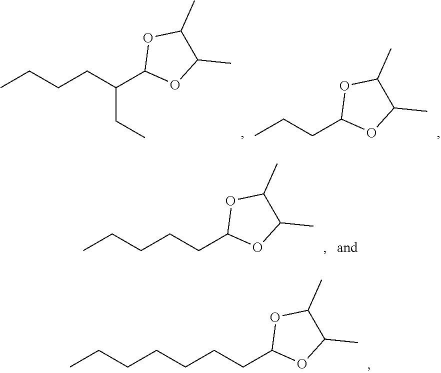

wherein said compound of Formula I is contacted with said diesel fuel in a ratio of 1:10, 2:10, or 3:10 (compound of formula I: diesel fuel), and wherein said property is cloud point, and said compound of Formula I improves said cloud point of said diesel fuel by at least 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%.

In certain embodiments, the subject matter disclosed herein is directed to a method of improving a property of a diesel fuel, comprising contacting a diesel fuel with a compound of Formula I, wherein said compound of Formula I is

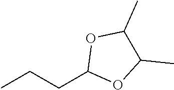

wherein said compound of Formula I is contacted with said diesel fuel in a ratio of 3:10 (compound of formula I: diesel fuel), wherein said property is cloud point, and said compound of Formula I improves said cloud point of said diesel fuel by at least 20%, 21%, 22%, 23%, 24%, 25%, 26%, 27%, 28%, 29%, 30%, 31%, 32%, 33%, 34%, 35%, 36%, 37%, 38%, 39%, or 40%.

In certain embodiments, R¹ is C₂ alkyl and R² is C₂ alkyl. In certain embodiments, R¹ is C₄ alkyl and R² is C₂ alkyl. In certain embodiments, R¹ is C₂ alkyl and R² is H. In certain embodiments, R¹ is C₄ alkyl and R² is H. In certain embodiments, R¹ is C₆ alkyl and R² is H.

In certain embodiments, the diesel fuel additive compound of Formula I is selected from the group consisting of

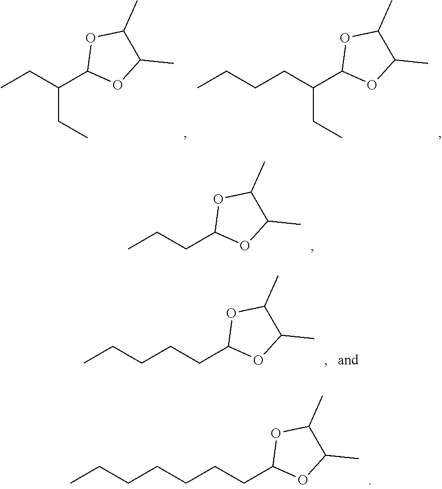

In certain embodiments, the subject matter disclosed herein is directed to a diesel fuel composition comprising a diesel fuel and a compound of Formula I,

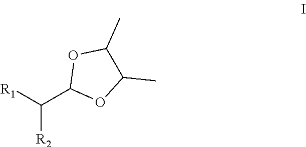

wherein R₁ is C₁₋₈ alkyl or C₂₋₆ alkyl and R₂ is selected from the group consisting of H, C₁₋₈ alkyl, and C₂₋₆ alkyl.

In certain embodiments, the diesel fuel is biodiesel fuel.

In certain embodiments, the diesel fuel is petroleum diesel fuel.

In certain embodiments, the compound of Formula I is mixed with diesel fuel in approximately a 1:10 ratio. In certain embodiments, the compound of Formula I is mixed with diesel fuel in approximately a 2:10 ratio, 3:10 ratio, 4:10 or 5:10 ratio. In certain embodiments, the diesel fuel is blended with a compound of Formula I in a ratio of about 1:10, 2:10, or 3:10 (compound of Formula I: diesel fuel).

In certain embodiments of the diesel fuel comprising a compound of Formula I, contacting the diesel fuel with a material does not cause swelling of said material. As used herein, “swelling” refers to a significant volume change (i.e. greater than 15%) upon an increase in the mass concentration of the dioxolane in ULSD. In certain embodiments, the material is selected from the group consisting of viton, flouorsilicones, neoprene, silicone, styrene-butadiene rubber (SBR), Ethylene Propylene Diene Monomer (EPDM) rubber, polyurethane, epichlorohydrin rubber (ECO), Paracril OZO plastic, hydrogenated acrylonitrile butadiene rubber (HNBR) and nitrile rubber. In certain embodiments, the compound of Formula I is

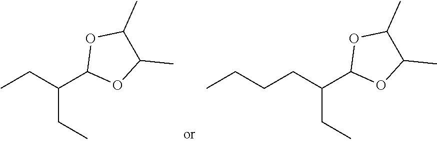

and the material is selected from the group consisting of viton, flouorsilicones, neoprene, silicone, styrene-butadiene rubber (SBR), Ethylene Propylene Diene Monomer (EPDM) rubber, polyurethane, epichlorohydrin rubber (ECO), Paracril OZO plastic, hydrogenated acrylonitrile butadiene rubber (HNBR) and nitrile rubber. In certain embodiments, the nitrile rubber is selected from the group consisting of NBR1, NBR2, NBR3, NBR4, NBR5, and NBR6.

In certain embodiments, R¹ is C₂ alkyl and R² is C₂ alkyl. In certain embodiments, R¹ is C₄ alkyl and R² is C₂ alkyl. In certain embodiments, R¹ is C₂ alkyl and R² is H. In certain embodiments, R¹ is C₄ alkyl and R² is H. In certain embodiments, R¹ is C₆ alkyl and R² is H.

In certain embodiments, the compound of Formula I is selected from the group consisting of

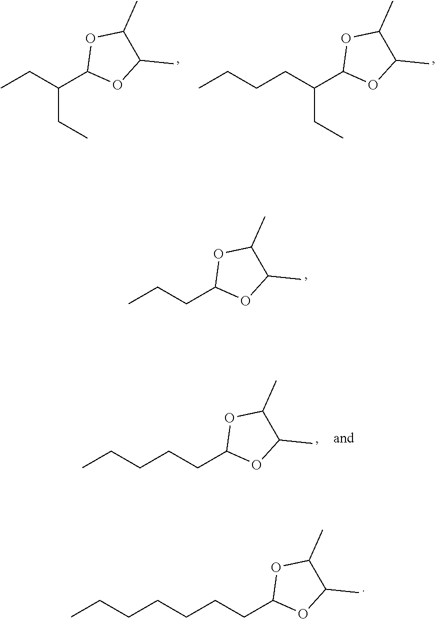

### III. Schemes

In certain embodiments, the methods described herein are directed to preparing 2-(1-Ethylpropyl)-4,5-dimethyl-1,3-dioxolane (1) in high yield with high purity. Scheme 1 depicts an overview for such a synthesis.

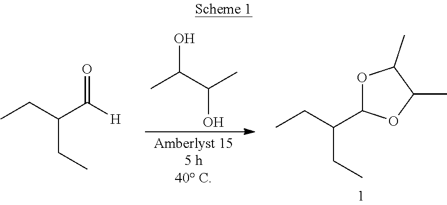

In certain embodiments, the methods described herein are directed to preparing 2-(1-Ethylpentyl)-4,5-dimethyl-1,3-dioxolane (2) in high yield, with high purity. Scheme 2 depicts an exemplary route for such a synthesis.

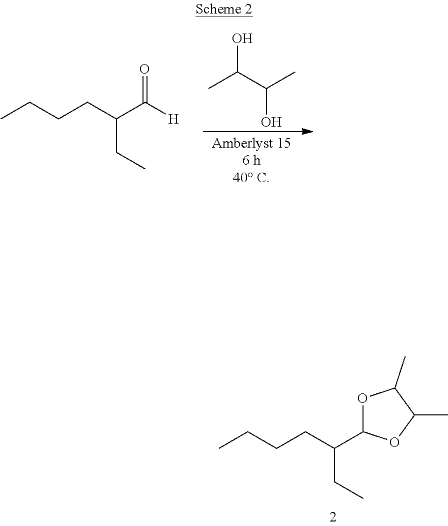

In certain embodiments, the methods described herein are directed to preparing 4,5-Dimethyl-2-propyl-1,3-dioxolane (3). Scheme 3 depicts an exemplary route for such a synthesis. In embodiments, NAFION can take the place of AMBERLYST 15.

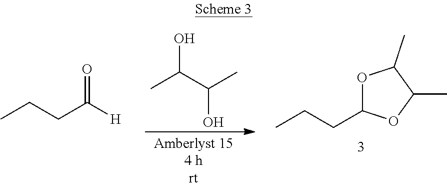

In certain embodiments, the methods described herein are directed to preparing 4,5-Dimethyl-2-pentyl-1,3-dioxolane (4). Scheme 4 depicts an exemplary route for such a synthesis.

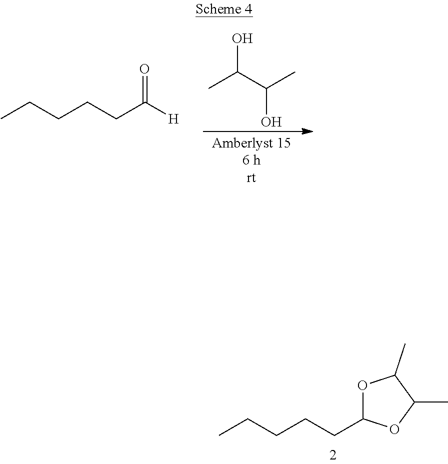

In certain embodiments, the methods described herein are directed to preparing 4,5-Dimethyl-2-heptyl-1,3-dioxolane (5). Scheme 5 depicts an exemplary route for such a synthesis.

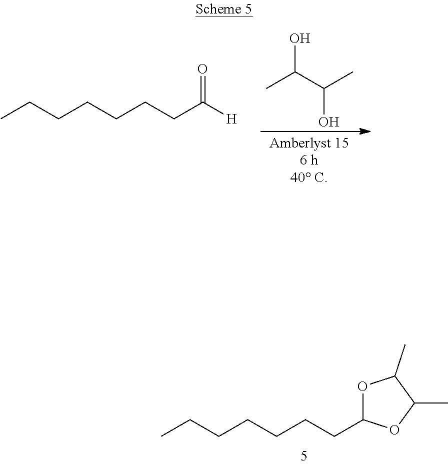

The subject matter described herein includes the following embodiments:

1. A process for the preparation of a diesel fuel additive compound of Formula I,

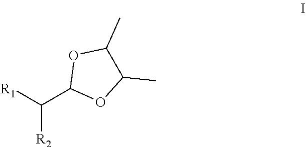

wherein R₁ is C₁₋₈ alkyl or C₂₋₆ alkyl and R₂ is selected from the group consisting of H, C₁₋₈ alkyl, and C₂₋₆ alkyl,

the process comprising:

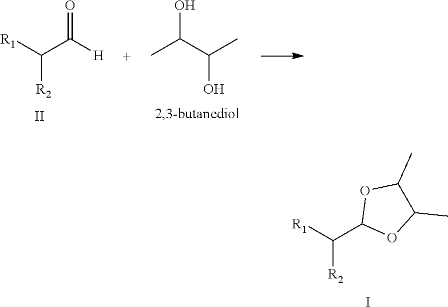

contacting a compound of formula II with 2,3-butanediol in the presence of a catalyst to form a solution;

heating said solution at a temperature of about 40° C.; and allowing said solution to separate into an upper phase and a lower phase.

2. The process of embodiment 1, further comprising separating the upper phase from said solution, wherein the upper phase comprises the compound of Formula I.
3. The process of embodiment 1 or 2, further comprising repeating said contacting, wherein said 2,3-butanediol is unreacted 2,3-butanediol from the lower phase.
4. The process of any one of embodiments 1-3, wherein the catalyst is Amberlyst 15.
5. The process of any one of embodiments 1-3, wherein the catalyst is NAFION.
6. The process of any one of embodiments 1-5, wherein heating is carried out between about 4 hours and about 6 hours.
7. The process of any one of embodiments 1-6, wherein the 2,3-butanediol is present in an amount ranging from about 1.25 to about 2.75 moles relative to 1 mole of the compound of Formula II.
8. The process of any one of embodiments 1-7, wherein, R¹ is selected from the group consisting of C₂ alkyl, C₄ alkyl, and C₆ alkyl; and R² is C₂ alkyl or H.
9. The process of any one of embodiments 1-8, wherein said diesel fuel additive compound of Formula I is selected from the group consisting of

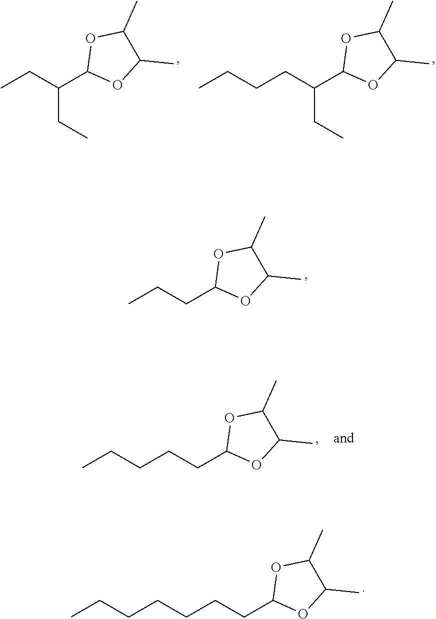

10. The process of any one of embodiments 1-9, wherein said diesel fuel additive compound is

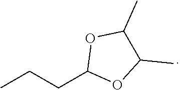

11. A method of improving a property of a diesel fuel, comprising:
contacting a diesel fuel with a compound of Formula I,

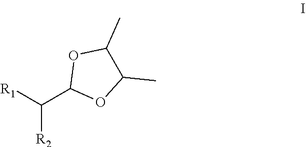

wherein R₁ is C₁₋₈ alkyl or C₂₋₆ alkyl and R₂ is selected from the group consisting of H, C₁₋₈ alkyl, and C₂₋₆ alkyl,

wherein said property is selected from the group consisting of cetane number, freezing point, viscosity, yield sooting index, net heat of combustion, density, and cloud point.

11B. A method of improving a property of a diesel fuel, comprising:
contacting a diesel fuel with a compound of Formula I,

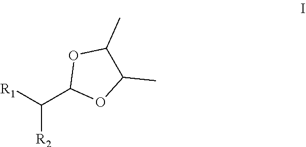

wherein R₁ is C₁₋₈ alkyl or C₂₋₆ alkyl and R₂ is selected from the group consisting of H, C₁₋₈ alkyl, and C₂₋₆ alkyl,

wherein said property is cloud point or cetane number.

12. The method of embodiment 11, wherein said property is viscosity and said compound of Formula 1 has a viscosity of about 0.9 mm² s⁻¹ to about 2.4 mm² s⁻¹ at 40° C.
13. The method of embodiment 11, wherein said property is cetane number and said compound of Formula 1 has a derived cetane number of about 45 to about 70.
14. The method of embodiment 11, wherein said property is freezing point and said compound of Formula I has a freezing point of less than −100° C.
15. The method of embodiment 11, wherein said property is density and said compound of Formula I has a density in a range of about 0.880 g/mL to about 0.895 g/mL at 25° C.
16. The method of embodiment 11, wherein said property is net heat of combustion and said compound of Formula I exhibits a net heat of combustion of about 27.60 to about 30.50 MJ/L.
17. The method of embodiment 11, wherein said property is yield sooting index, wherein said compound of Formula I exhibits a yield sooting index of about 35 to about 70.
18. The method of any one of embodiments 11-17, wherein R¹ is selected from the group consisting of C₂ alkyl, C₄ alkyl, and C₆ alkyl and R² is C₂ alkyl or H.
19. The method of any one of embodiments 11-18, wherein said compound of Formula I is selected from the group consisting of

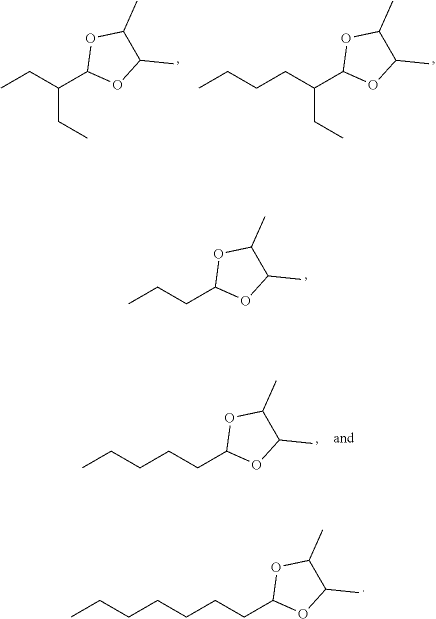

20. A diesel fuel composition comprising a diesel fuel and a compound of Formula I,

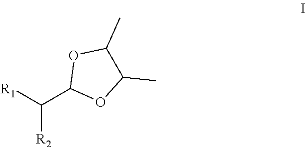

wherein R₁ is C₁₋₈ alkyl or C₂₋₆ alkyl and R₂ is selected from the group consisting of H, C₁₋₈ alkyl, and C₂₋₆ alkyl.
21. The diesel fuel composition of embodiment 20, wherein said diesel fuel is biodiesel fuel.
22. The diesel fuel composition of embodiment 20, wherein said diesel fuel is petroleum diesel fuel.
23. The diesel fuel composition of any one of embodiments 20-22, wherein said compound is mixed with said diesel fuel in approximately a 1:10 to 5:10 ratio.
24. The diesel fuel composition of embodiment 23, wherein said compound is mixed with said diesel fuel in approximately a 1:10 ratio.
25. The diesel fuel composition of embodiment 23, wherein said compound is mixed with said diesel fuel in approximately a 2:10 ratio.
26. The diesel fuel composition of embodiment 23, wherein said compound is mixed with said diesel fuel in approximately a 3:10 ratio.
27. The diesel fuel composition of any one of embodiments 20-26, wherein contacting the diesel fuel composition with a material does not cause swelling of said material.
28. The diesel fuel composition of embodiment 27, wherein the material is selected from the group consisting of viton, flouorsilicones, neoprene, silicone, styrene-butadiene rubber (SBR), Ethylene Propylene Diene Monomer (EPDM) rubber, polyurethane, epichlorohydrin rubber (ECO), Paracril OZO plastic, hydrogenated acrylonitrile butadiene rubber (HNBR) and nitrile rubber.
29. The diesel fuel composition of embodiment 28, wherein said nitrile rubber is selected from the group consisting of NBR1, NBR2, NBR3, NBR4, NBR5, and NBR6.
30. The diesel fuel composition of any one of embodiments 20-29, wherein R¹ is selected from the group consisting of C₂ alkyl, C₄ alkyl, and C₆ alkyl; and R² is C₂ alkyl or H.
31. The diesel fuel composition of any one of embodiments 20-30, wherein said compound of Formula I is selected from the group consisting of

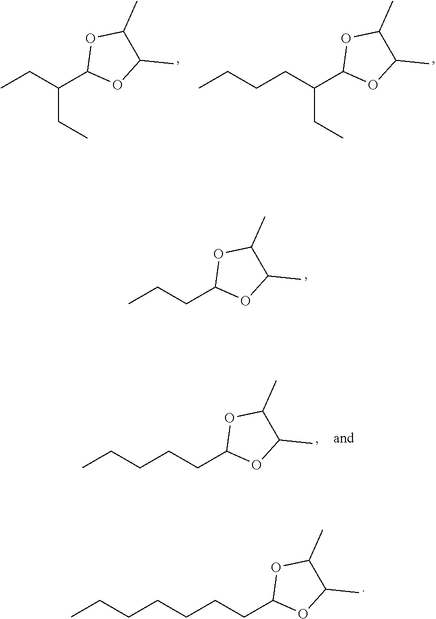

The present invention is further described in the following non-limiting Examples. It should be understood that these Examples, while indicating preferred embodiments of the invention, are given by way of illustration only.

### EXAMPLES

### Materials and Methods

All chemicals and reagent grade solvents were obtained from commercial vendors and were used as received. ¹H and ¹³C NMR spectra were obtained at room temperature on a Bruker AV400 MHz spectrometer, with chemical shifts (6) referenced to the residual solvent signal (¹H and ¹³C). GC/MS analysis was carried out using a Hewlett-Packard 6890 GC system equipped with a Hewlett-Packard 5973 mass selective detector. FT-IR measurements were collected on a Perkin Elmer Spectrum Two FT-IR, where a drop of compound was pressed between two KBr plates. FTIR shifts were: very strong (vs), strong (s), medium (m), and weak (w). Combustion calorimeter measurements were performed using an IKA C1 compact combustion calorimeter, the NHOC was calculated by taking into consideration the hydrogen content and subtracting from the gross heat of combustion. DSC measurements were performed using a Netzsch DCS 204 F1 Phoenix. Viscosity and density measurements were performed using an Anton Paar SVM 3001. Elemental analysis was performed by Atlantic Microlabs. YSI measurements were conducted at Yale University.

Calculation of Atom Economy and Carbon Yield

The following equation was used to determine the atom economy:

$$atom economy = \frac{( mass of atoms in desired product )}{( mass of atoms in reactants )} * 100$$

The following equation was used to determine the carbon yield:

$$\% Carbon yield = \frac{( mmol carbon in product ) * ( isolated yield )}{( mmol carbon in reactant )} * 100$$

Note that the only side product in each reaction was water, so the carbon efficiency was 100% for every reaction; therefore, the carbon yield was the same as the isolated yield of the analytically pure product.

### Example 1: Preliminary Acetalization Investigations with BDO

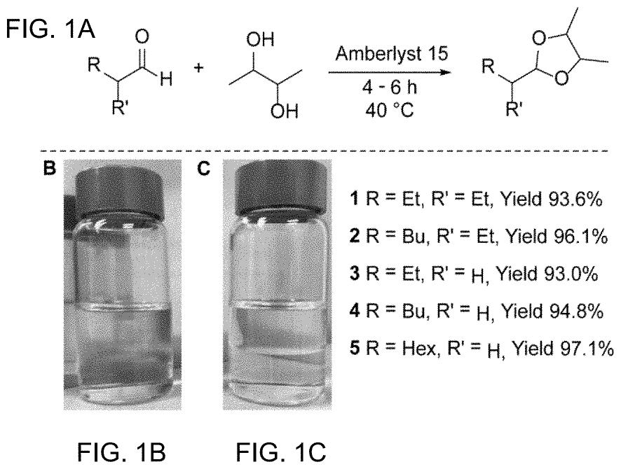

The acetalization of ethanol-derived C₄₋₈ aldehydes with BDO was investigated using the solid acid catalyst, Amberlyst 15. Initially, the reaction of 2-ethylbutyraldehyde and BDO was investigated by stirring with Amberlyst 15 in the absence of any solvent. The synthesis is outlined in FIG. 1A, where the reaction was conducted in a slight BDO molar excess (ca. 1.5:1; V/V). The two compounds were miscible when mixed, as shown in FIG. 1B. After stirring for 5 h under mild (40° C.) temperatures, the reaction was complete and a distinct phase separation was observed, FIG. 1C. The organic phase contained only the desired 1,3-dioxolane while the unreacted BDO and the produced water formed an aqueous layer. This phase separation, along with the absence of any solvent, allowed the reaction product to be decanted and the remaining BDO could then be used for additional cycles, with no additional workup or purification. The initial reaction of 2-ethylbutyraldehyde and BDO yielded compound 1, in 93.6% isolated yield. The acetalization of BDO was then investigated with 2-ethylhexanal and butyraldehyde to obtain 2 (96.1% yield) and 3 (93.0% yield), respectively. Given the availability of both branched and straight chain aldehydes as starting materials, the effect of branching on cetane number was studied. To this end, hexanal and octanal were reacted with BDO to obtain the straight chain acetals 4 (94.8% yield) and 5 (97.1% yield), respectively.

Given that the only loss in atom economy was water, a quantitative conversion with atom economy greater than 88.9% was achieved for all five reactions, with carbon yields equaling the isolated yield. The purity of each of the acetals was determined via ¹H NMR, and shown to be a mixture of three diastereomers. In these spectra, no water was observed, suggesting low or zero solubility. As such, no toxic side products were identified and the water could be separated from the remaining BDO via distillation. Additionally, each reaction proceeded to completion, and while no carbon was lost in the form of unidentifiable side products, the carbon balance was 100% for each reaction.

### Example 2: Detailed Synthesis of 2-(1-Ethylpropyl)-4,5-dimethyl-1,3-dioxolane (1)

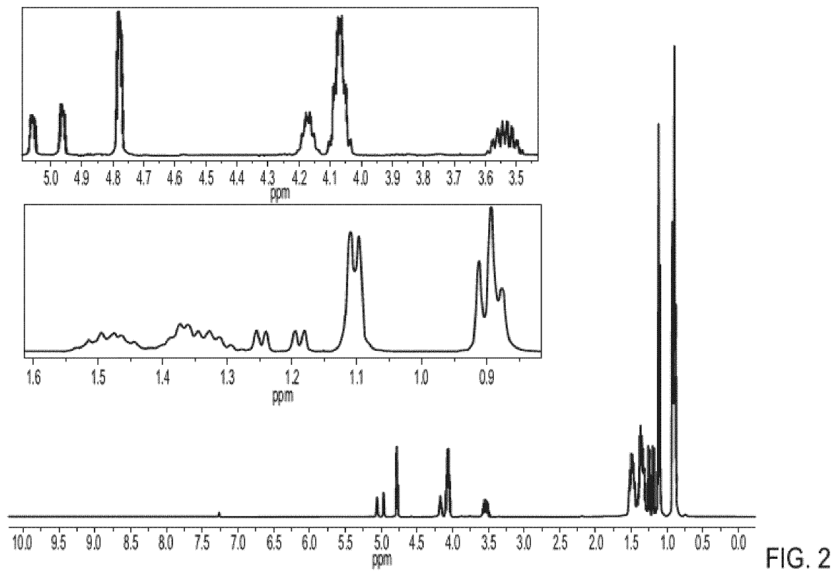

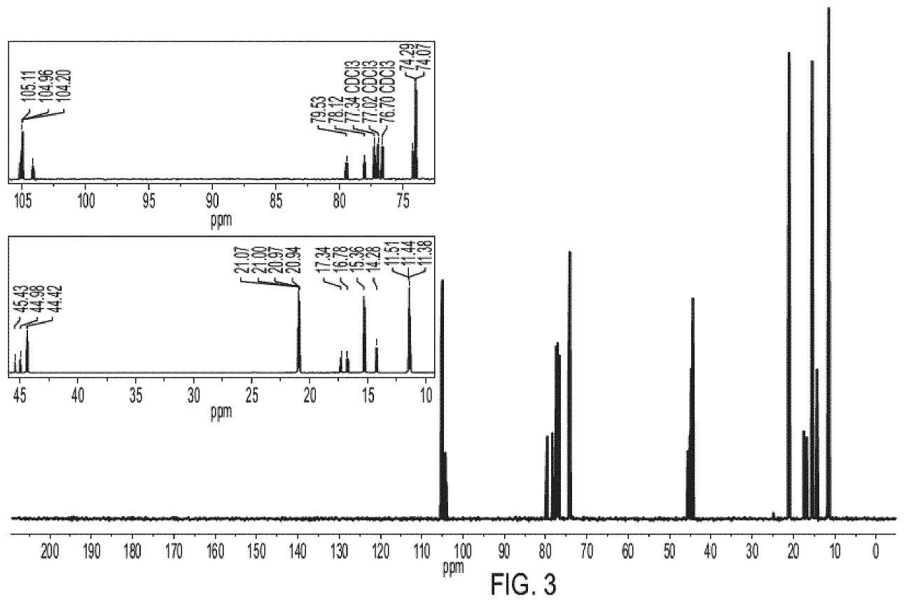

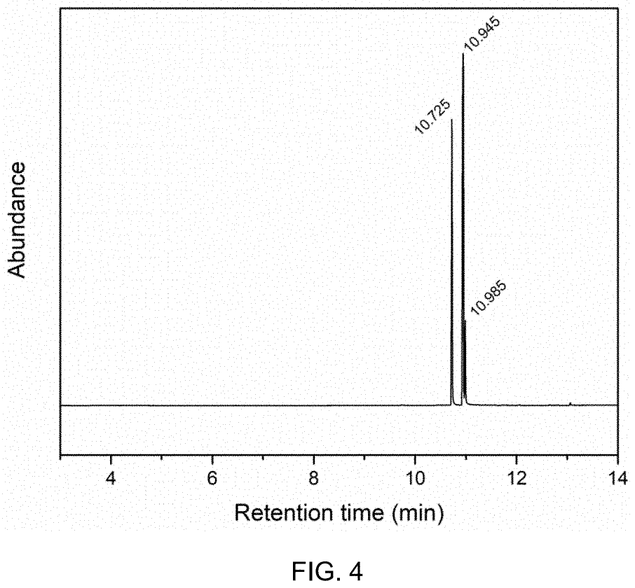

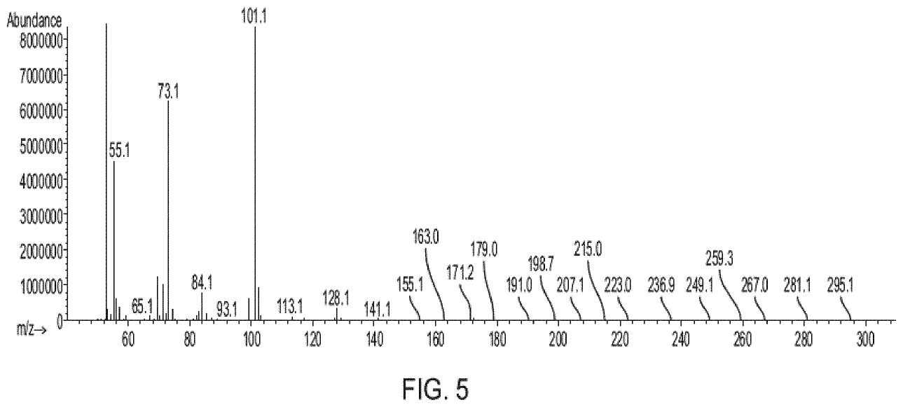

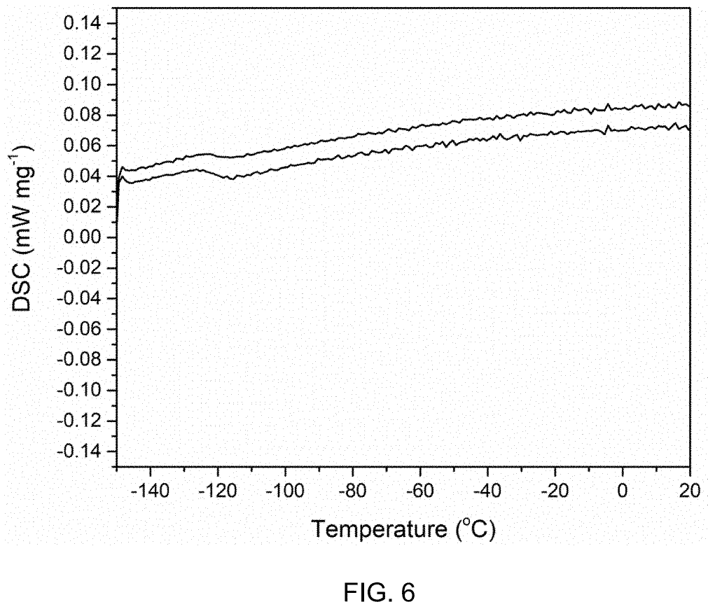

To prepare 2-(1-Ethylpropyl)-4,5-dimethyl-1,3-dioxolane, 2-Ethylbutyraldehyde (3.135 g, 31.30 mmol), 2,3-Butanediol (4.685 g, 46.77 mmol), and Amberlyst 15 (0.198 g) were combined in a 20 mL scintillation vial. The apparatus was heated to 40° C. with stirring (500 rpm). After 4 h the reaction was allowed to sit for 15 min, where two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield (1) as a mixture of three diastereomers (5.084 g, 96.6% isolated yield). NMR, GC, MS, and DSC spectra of (1) are provided in FIGS. 2-6.

¹H NMR (400 MHz, Chloroform-d) δ 5.06 (d, J=3.5 Hz, 0.2H), 4.96 (d, J=3.2 Hz, 0.2H), 4.78 (d, J=3.3 Hz, 0.6H), 4.17 (q, J=5.3 Hz, 0.4H), 4.07 (q, J=5.4 Hz, 1.2H), 3.54 (app. oct., J=6.6 Hz, 0.4H), 1.58-1.28 (m, 5H), 1.25 (d, J=5.7 Hz, 0.7H), 1.19 (d, J=5.7 Hz, 0.7H), 1.10 (d, J=5.2 Hz, 4.6H), 0.89 (app. t, J=7.2 Hz, 6H). Note: Fractional hydrogen integrations represent diastereomeric ratios.

¹³C NMR (101 MHz, Chloroform-d) δ 105.11, 104.96, 104.20, 79.53, 78.12, 74.29, 74.07, 45.43, 44.98, 44.42, 24.86, 21.07, 21.00, 20.97, 20.94, 17.34, 16.78, 15.36, 14.28, 11.51, 11.44, and 11.38.

FTIR (cm⁻¹): 2966 (vs), 2937 (s), 2877 (s), 1458 (m), 1419 (w), 1380 (m), 1339 (w), 1182 (w), 1105 (vs), 1038 (m), 983 (w), and 924 (w).

Anal. Calcd for C₁₀H₂₀O₂: C, 69.72; H, 11.70. Found: C, 69.61; H, 11.80.

### Example 3: Detailed Synthesis of 2-(1-Ethylpentyl)-4,5-dimethyl-1,3-dioxolane (2)

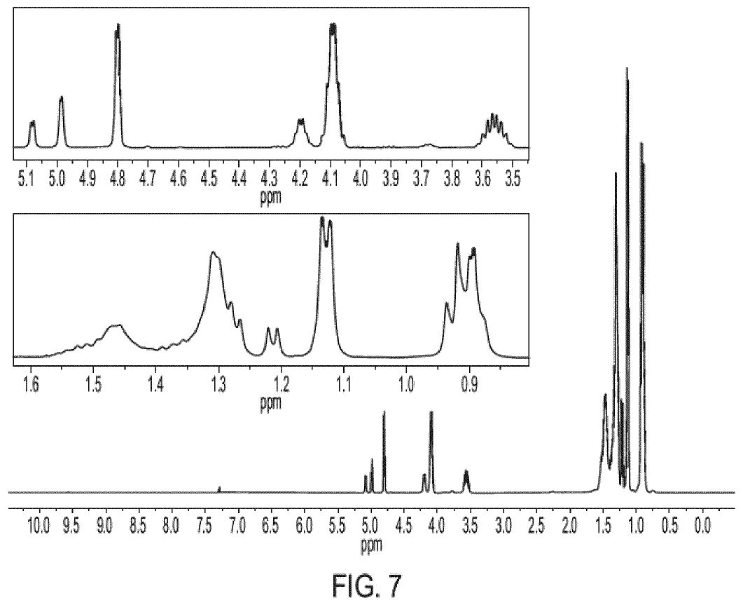

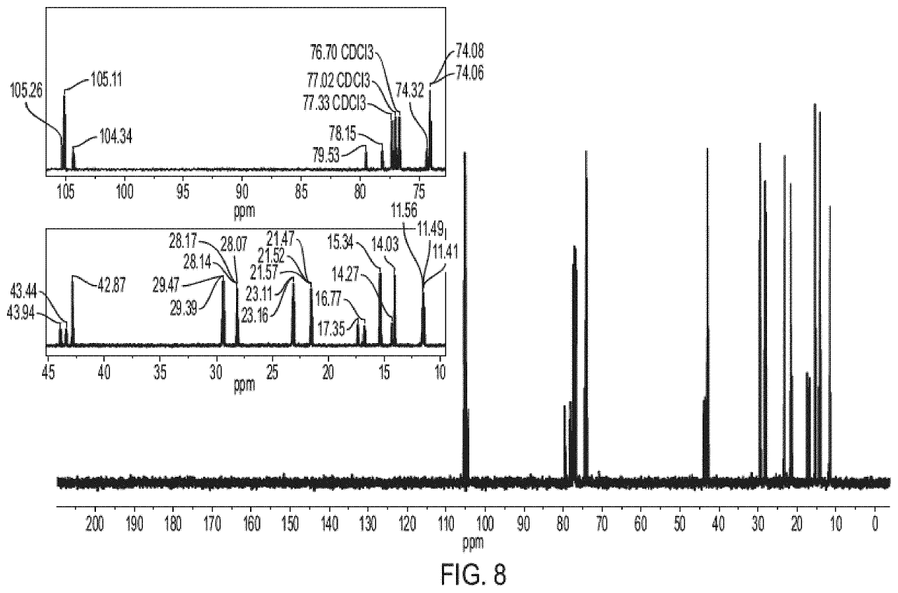

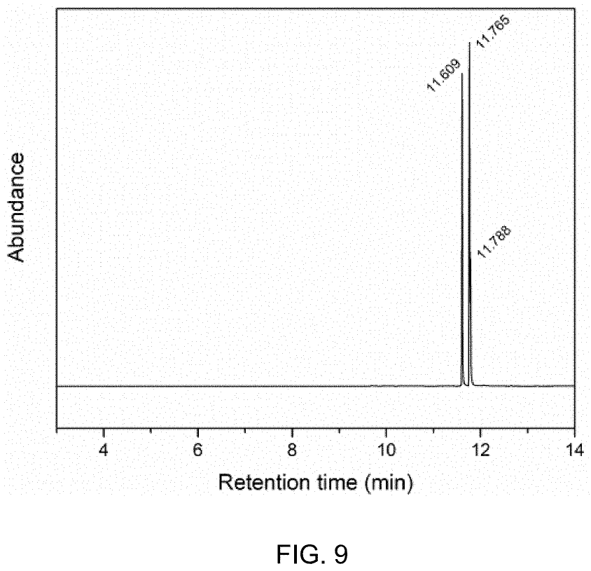

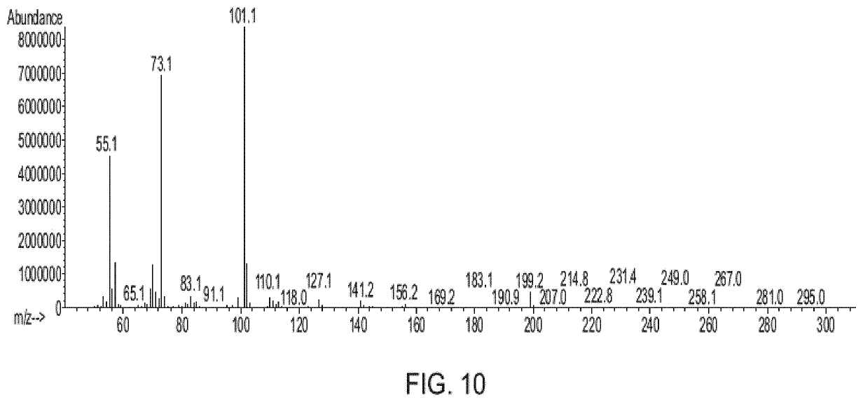

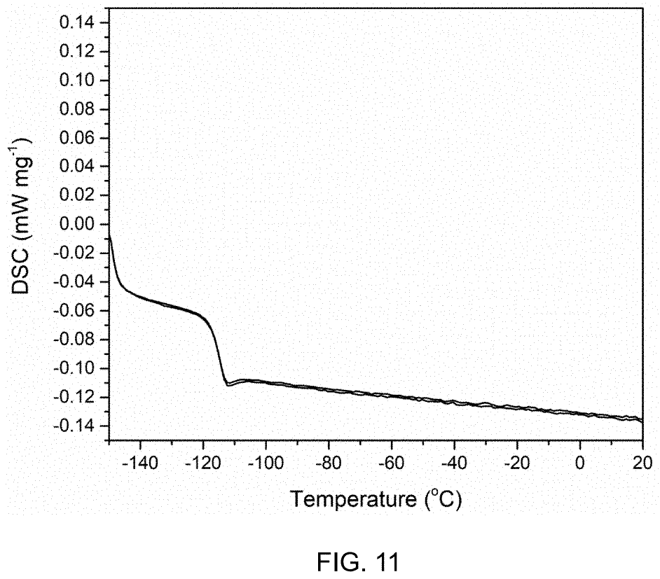

To prepare 2-(1-Ethylpentyl)-4,5-dimethyl-1,3-dioxolane (2), 2-Ethylhexanal (4.346 g, 33.89 mmol), 2,3-Butanediol (6.030 g, 60.20 mmol), and Amberlyst 15 (0.201 g) were combined in a 20 mL scintillation vial. The apparatus was heated to 40° C. with stirring (500 rpm). After 6 h the reaction was allowed to sit for 15 min, where two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield 2 as a mixture of three diastereomers (6.520 g, 96.1% isolated yield). NMR, GC, MS, and DSC spectra of (2) are provided in FIGS. 7-11.

¹H NMR (400 MHz, Chloroform-d) δ 5.08 (d, J=3.8 Hz, 0.2H), 4.99 (d, J=2.9 Hz, 0.2H), 4.80 (d, J=3.1 Hz, 0.6H), 4.20 (q, J=5.1 Hz, 0.3H), 4.09 (h, J=6.4 Hz, 1.2H), 3.56 (app. oct., J=6.5 Hz, 0.5H), 1.59-1.24 (m, 9.5H), 1.21 (d, J=5.7 Hz, H), 1.13 (d, J=5.2 Hz, 4.5H), 0.91 (m, J=9.2, 6.5 Hz, 6H). Note: Fractional hydrogen integrations represent diastereomeric ratios.

¹³C NMR (101 MHz, Chloroform-d) δ 105.26, 105.11, 104.34, 79.53, 78.15, 74.32, 74.08, 74.32, 74.08, 74.06, 43.94, 43.44, 42.87, 29.47, 29.39, 28.17, 28.14, 28.07, 23.16, 23.11, 21.57, 21.52, 21.47, 17.35, 16.77, 15.34, 14.27, 14.03, 11.56, 11.49, and 11.41.

FTIR (cm⁻¹): 2960 (vs), 2931 (vs), 2873 (vs), 1457 (m), 1378 (m), 1340 (w), 1309 (w), 1179 (w), 1108 (vs), 1039 (m), 1000 (w), and 972 (w).

Anal. Calcd for C₁₂H₂₄O₂: C, 71.95; H, 12.08. Found: C, 71.01; H, 12.35.

### Example 4A: Detailed Synthesis of 4,5-Dimethyl-2-propyl-1,3-dioxolane (3) Using Amberlyst 15

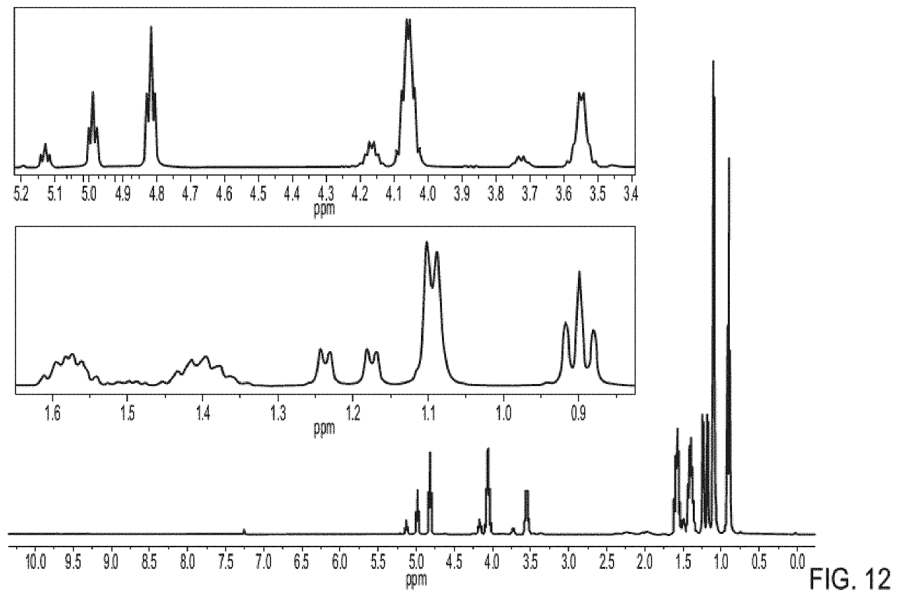

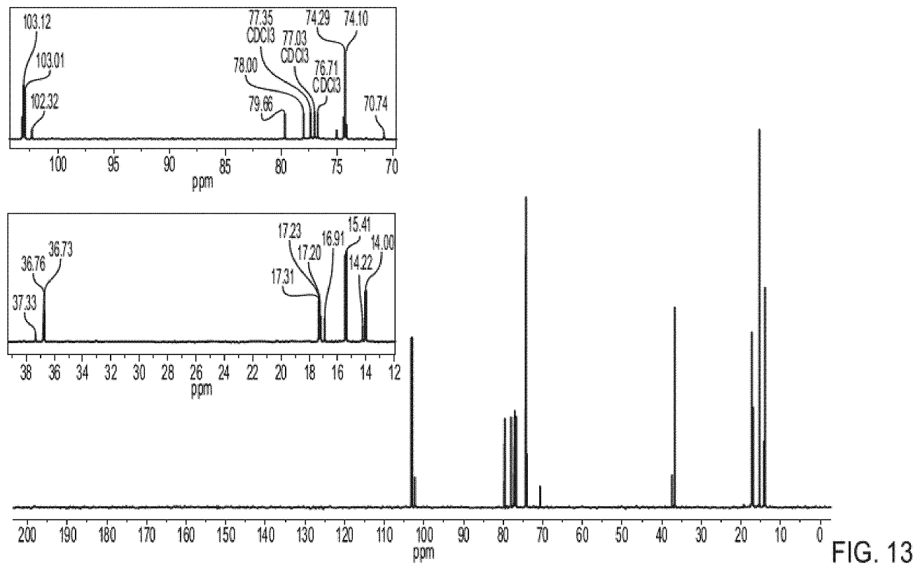

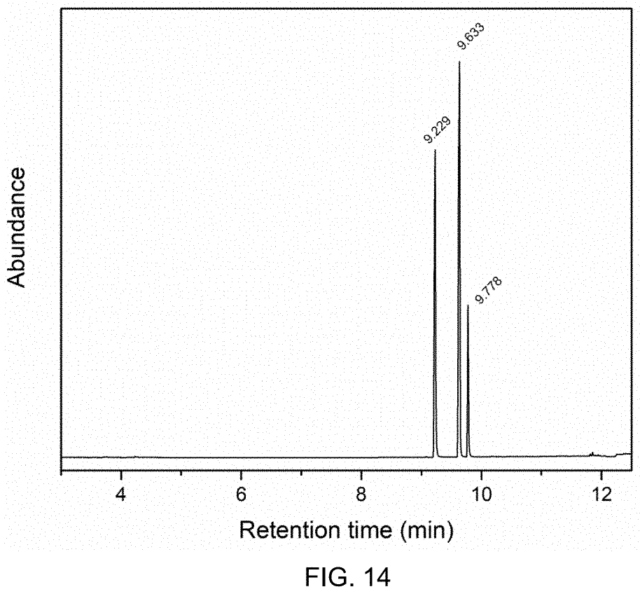

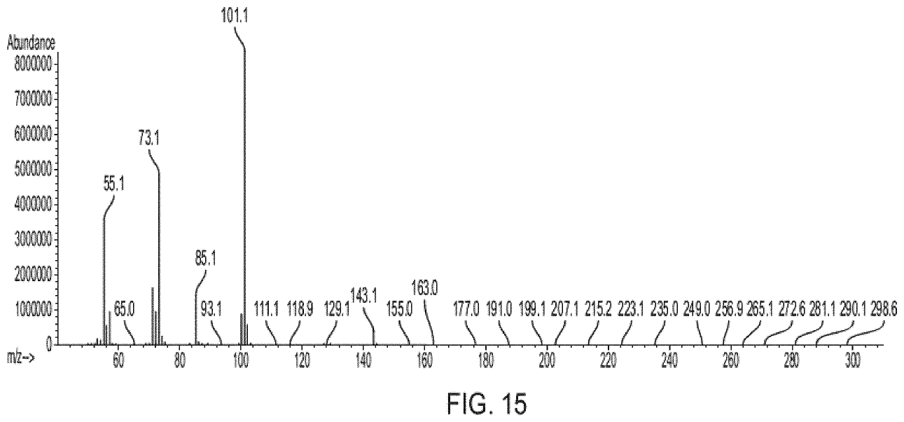

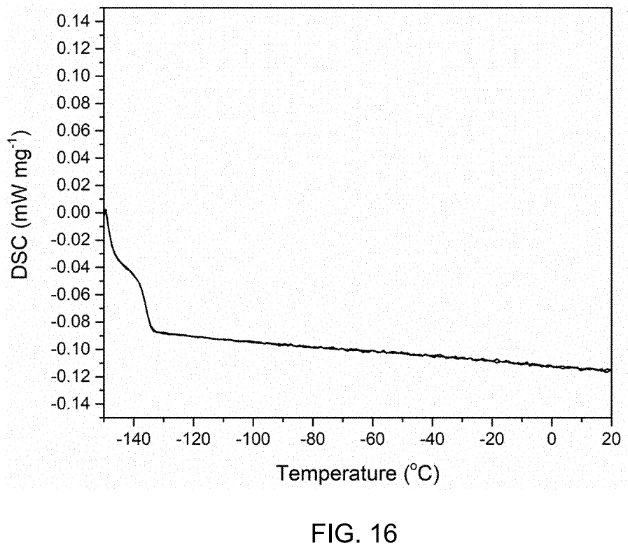

To prepare 4,5-Dimethyl-2-propyl-1,3-dioxolane (3), butyraldehyde (4.028 g, 55.86 mmol), 2,3-Butanediol (8.028 g, 89.08 mmol), and Amberlyst 15 (0.401 g) were combined in a 20 mL scintillation vial. The apparatus was stirred (500 rpm) at room temperature. After 4 h the reaction was allowed to sit for 15 min, where two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield 3 as a mixture of three diastereomers (7.494 g, 93.0% isolated yield). NMR, GC, MS, and DSC spectra of (3) are provided in FIGS. 12-16.

¹H NMR (400 MHz, Chloroform-d) δ 5.13 (t, J=4.9 Hz, 0.1H), 4.99 (t, J=4.7 Hz, 0.3H), 4.82 (t, J=4.7 Hz, 0.6H), 4.17 (p, J=5.3 Hz, 0.2H), 4.06 (h, J=6.3 Hz, 1.1H), 3.73 (d, J=5.4 Hz, 0.1H), 3.55 (q, J=6.0 Hz, 0.6H), 1.66-1.32 (m, 4H), 1.24 (d, J=5.0 Hz, 2H), 1.17 (d, J=5.1 Hz, 2H), 1.09 (d, J=5.4 Hz, 4H), 0.90 (t, J=7.4 Hz, 3H). Note: Fractional hydrogen integrations represent diastereomeric ratios.

¹³C NMR (101 MHz, Chloroform-d) δ 103.12, 103.01, 102.32, 79.66, 78.00, 74.29, 74.10, 70.74, 37.33, 36.76, 36.73, 17.31, 17.23, 17.20, 16.91, 15.41, 14.22, and 14.00.

FTIR (cm¹): 2962 (vs), 2935 (vs), 2874 (vs), 1459 (m), 1378 (s), 1322 (w), 1234 (w), 1147 (s), 1120 (vs), 1032 (w), 983 (w), 957 (m), 911 (m), and 837 (w).

Anal. Calcd for C₈H₁₆O₂: C, 66.63; H, 11.18. Found: C, 66.17; H, 11.41.

### Example 4B: Detailed Synthesis of 4,5-Dimethyl-2-propyl-1,3-dioxolane (3) Using Nafion

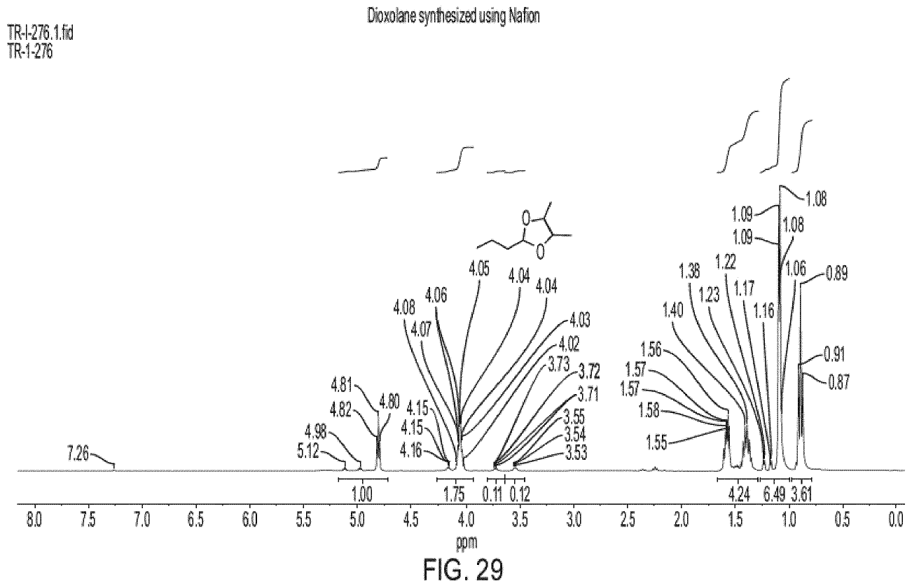

To prepare 4,5-Dimethyl-2-propyl-1,3-dioxolane (3), butyraldehyde (4.028 g, 55.86 mmol), 2,3-Butanediol (8.028 g, 89.08 mmol), and Nafion 15 (0.401 g) were combined in a 20 mL scintillation vial. The apparatus was stirred (500 rpm) at room temperature. After 4 h the reaction was allowed to sit for 15 min, where two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield 3 as a mixture of three diastereomers (7.494 g, 93.0% isolated yield). An NMR spectrum is provided in FIG. 29.

### Example 5: Detailed Synthesis of 4,5-Dimethyl-2-pentyl-1,3-dioxolane (4)

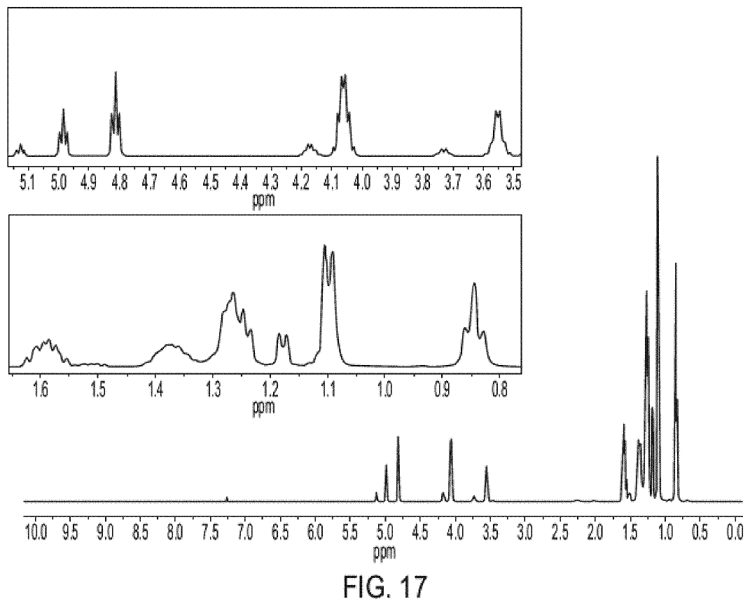

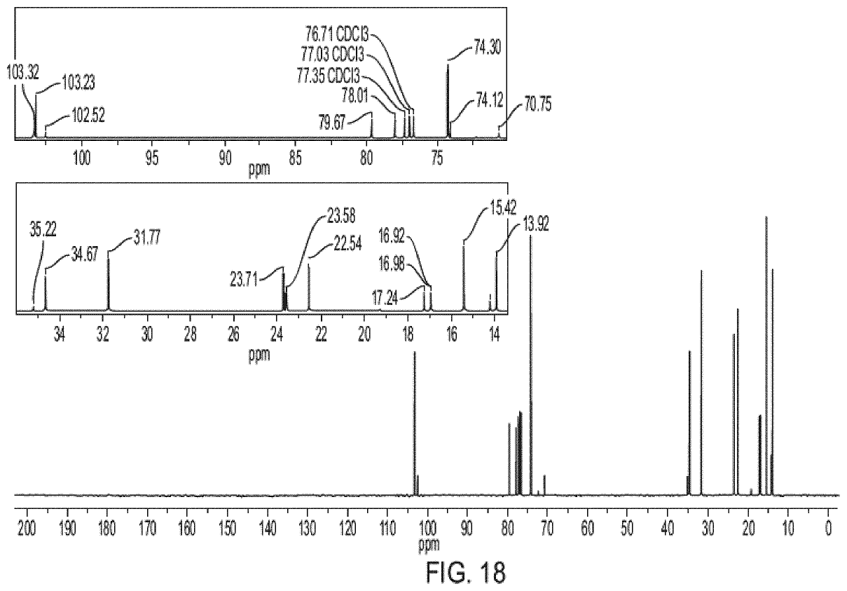

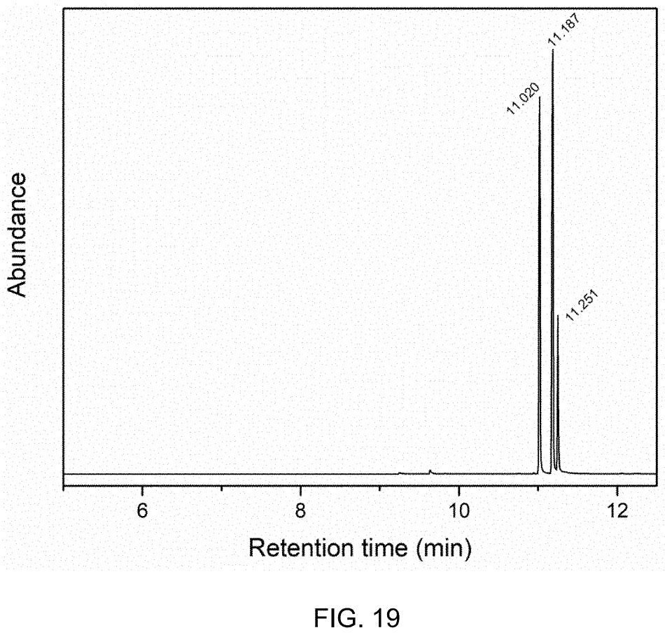

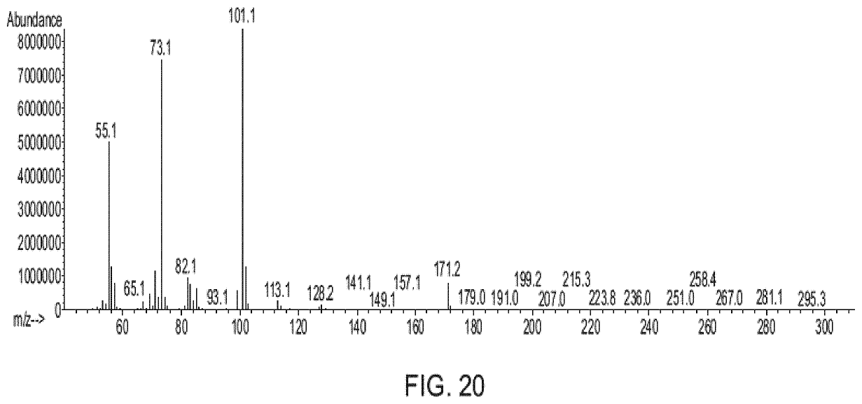

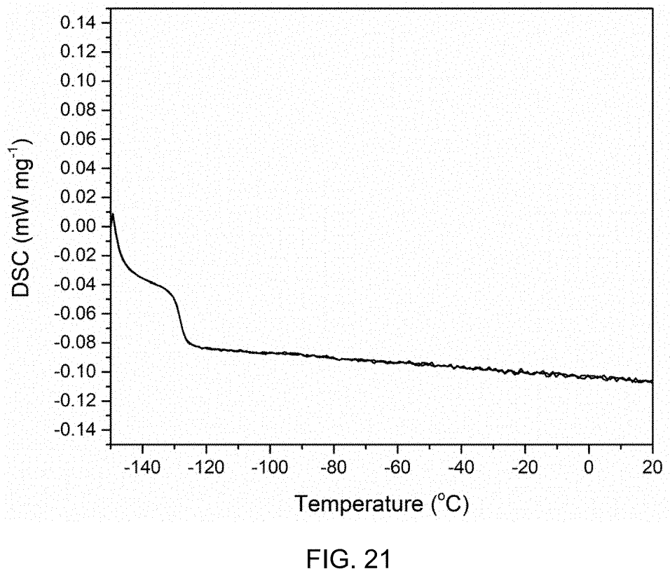

To prepare 4,5-Dimethyl-2-pentyl-1,3-dioxolane (4), hexanal (4.029 g, 40.22 mmol), 2,3-Butanediol (7.772 g, 86.24 mmol), and Amberlyst 15 (0.413 g) were combined in a 20 mL scintillation vial. The apparatus was stirred (500 rpm). After 4 h the reaction was allowed to sit for 15 min, where two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield 4 as a mixture of three diastereomers (6.571 g, 94.8% isolated yield). NMR, GC, MS, and DSC spectra of (4) are provided in FIGS. 17-21.

¹H NMR (400 MHz, Chloroform-d) δ 5.13 (t, J=5.0 Hz, 0.1H), 4.98 (t, J=4.8 Hz, 0.3H), 4.81 (t, J=4.8 Hz, 0.6H), 4.16 (p, J=5.4 Hz, 0.2H), 4.06 (h, J=6.3 Hz, 1.1H), 3.73 (q, J=5.6 Hz, 0.1H), 3.55 (dd, J=7.7, 4.6 Hz, 0.6H), 1.59 (qd, J=7.4, 5.0 Hz, 2H), 1.44-1.21 (m, 7H), 1.18 (d, J=5.1 Hz, 1H), 1.10 (d, J=5.3 Hz, 4H), 0.92-0.78 (m, 3H). Note: Fractional hydrogen integrations represent diastereomeric ratios.

¹³C NMR (101 MHz, Chloroform-d) δ 103.32, 103.23, 102.52, 79.67, 78.01, 74.30, 74.12, 70.75, 35.22, 34.67, 31.77, 23.71, 23.58, 22.54, 17.24, 16.98, 16.92, 15.42, 14.22, and 13.92. FTIR (cm-1): 2957 (vs), 2931 (vs), 2863 vs), 1460 (m), 1418 (w), 1378 (s), 1145 (s), 1118 (vs), 1082 (s), 1041 (m), 993 (w), 929 (m), and 889 (w).

Anal. Calcd for C10H20O2: C, 69.72; H, 11.70; 0. Found: C, 69.09; H, 11.77.

### Example 6: Detailed Synthesis of 4,5-Dimethyl-2-heptyl-1,3-dioxolane (5)

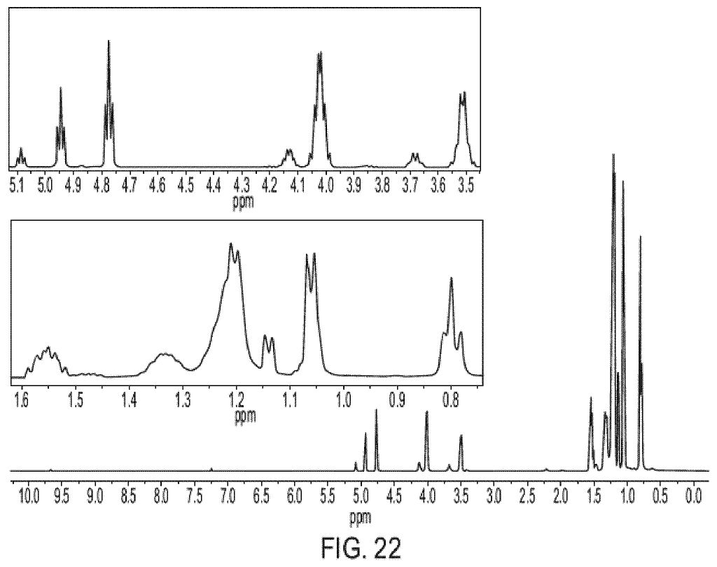

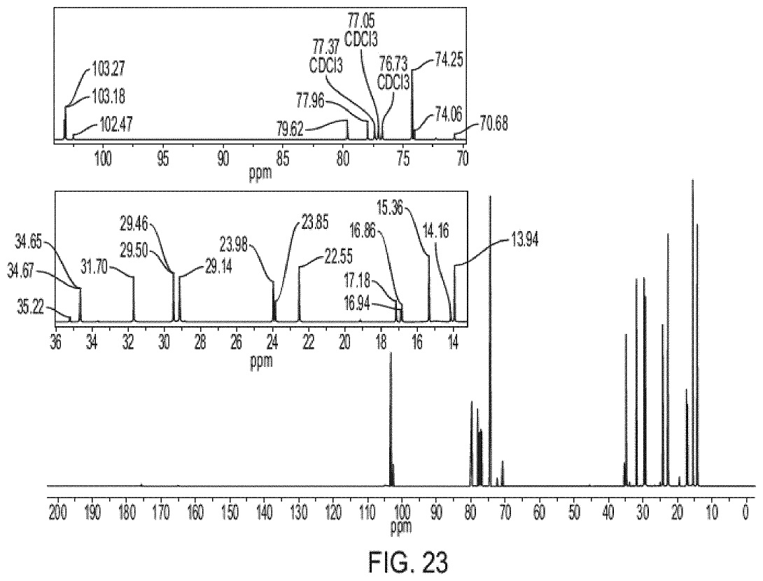

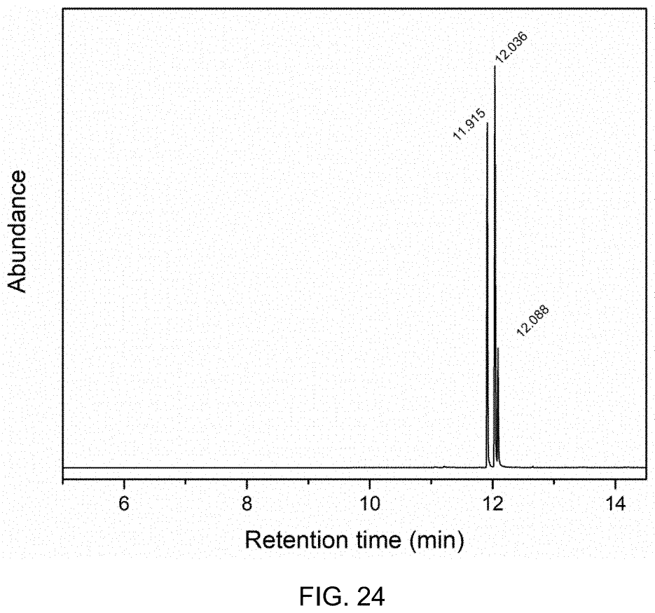

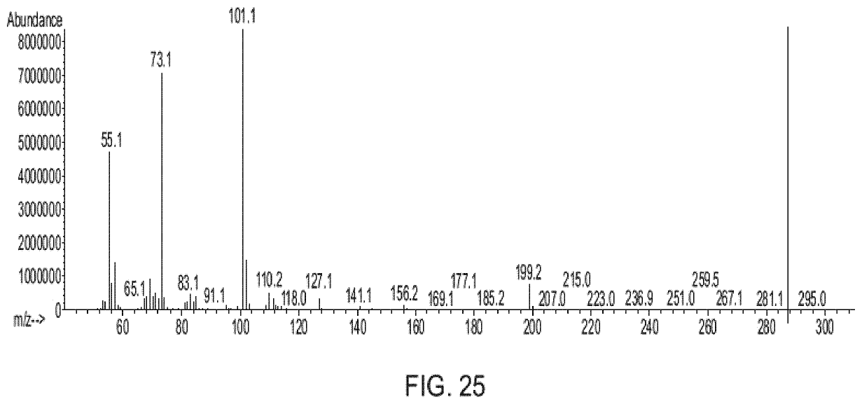

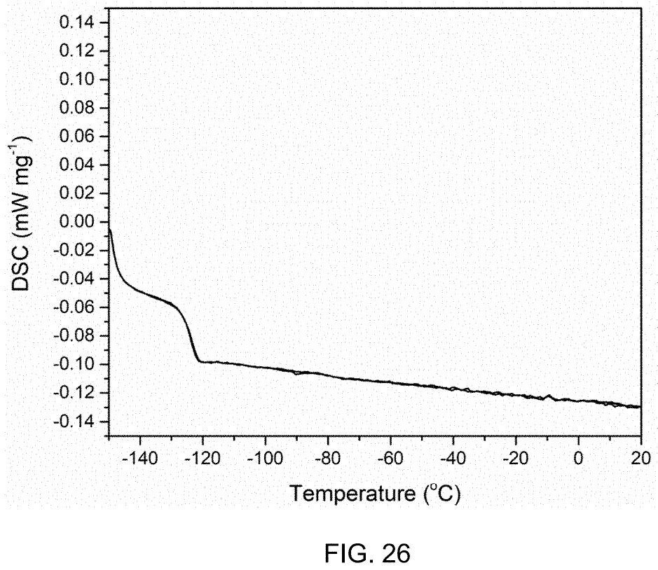

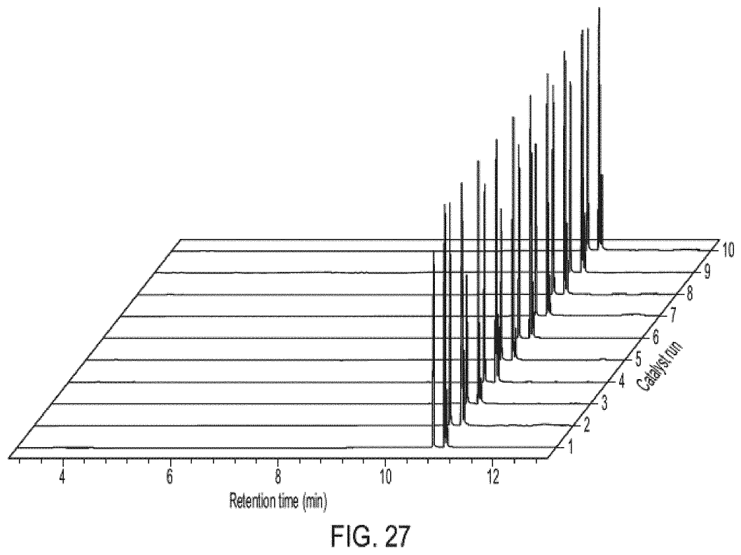

To prepare 4,5-Dimethyl-2-heptyl-1,3-dioxolane (5). Octanal (4.003 g, 31.22 mmol), 2,3-Butanediol (7.142 g, 79.25 mmol), and Amberlyst 15 (0.385 g) were combined in a 20 mL scintillation vial. The apparatus was heated to 40° C. with stirring (500 rpm). After 6 h the reaction was allowed to cool for 15 min. Upon cooling, two distinct layers formed, the top layer contained the reaction product while the bottom (aqueous) layer contained remaining 2,3-Butanediol and water. The organic phase was decanted and an aliqout diluted with methanol for GCMS to reveal >99% conversion to the desired product. An aliquot of the resulting liquid was analyzed by NMR to yield 5 as a mixture of three diastereomers (6.072 g, 97.1% isolated yield). NMR, GC, MS, and DSC spectra of (5) are provided in FIGS. 22-26.

¹H NMR (400 MHz, Chloroform-d) δ 5.09 (t, J=4.9 Hz, 0.1H), 4.95 (t, J=4.7 Hz, 0.4H), 4.77 (t, J=4.8 Hz, 0.5H), 4.13 (h, J=5.6 Hz, 0.2H), 4.02 (h, J=6.2 Hz, 1H), 3.68 (d, J=5.9 Hz, 0.1H), 3.51 (dd, J=7.7, 4.5 Hz, 0.7H), 1.55 (qd, J=7.4, 4.9 Hz, 1.8H), 1.47 (dd, J=9.1, 5.1 Hz, 0.2H), 1.38-1.12 (m, 12H), 1.06 (d, J=5.3 Hz, 4H), 0.80 (t, J=6.5 Hz, 3H). Note: Fractional hydrogen integrations represent diastereomeric ratios.

¹³C NMR (101 MHz, Chloroform-d) δ 103.27, 103.18, 102.47, 79.62, 77.96, 74.25, 74.06, 70.68, 35.22, 34.637, 34.65, 31.70, 29.50, 29.46, 29.14, 23.98, 23.85, 22.55, 17.18, 16.94, 16.85, 15.36, 14.16, and 13.94.

FTIR (cm⁻¹): 2957 (vs), 2931 (vs), 2866 (vs), 1463 (m), 1413 (w), 1379 (s), 1346 (w), 1320 (w), 1146 (vs), 1082 (s), 1041 (m), 998 (m), 929 (m), and 893 (w).

Anal. Calcd for C₁₂H₂₄O₂: C, 71.95; H, 12.08. Found: C, 71.77; H, 12.30.

### Example 7: Large Scale Reaction

A representative example of the scalability of the above reactions was performed as follows. 2-Ethylhexanal (20.066 g, 156.5 mmol), 2,3-Butanediol (29.248 g, 324.5 mmol), and Amberlyst 15 (1.624 g) were combined in a 100 mL round bottom flask. The apparatus was heated to 40° C. with stirring (500 rpm). After 15 h the reaction was allowed to cool for 15 min. Upon cooling, two distinct layers formed, the top layer contained the reaction product while the bottom (aqueous) layer contained remaining 2,3-Butanediol and water. The organic phase was decanted and an aliqout diluted with methanol for GCMS to reveal >98% conversion to the desired product. The remaining reaction mixture was washed with cyclohexane (3×3 mL) and the resultant organic phase again decanted. The organic phases were combined and dried over MgSO₄. The solvent was removed in vacuo and an aliquot of the resulting liquid was analyzed by NMR to yield 2 as a mixture of three diastereomers (30.490 g, 97.3% isolated yield).

### Example 8: Catalyst Recycling

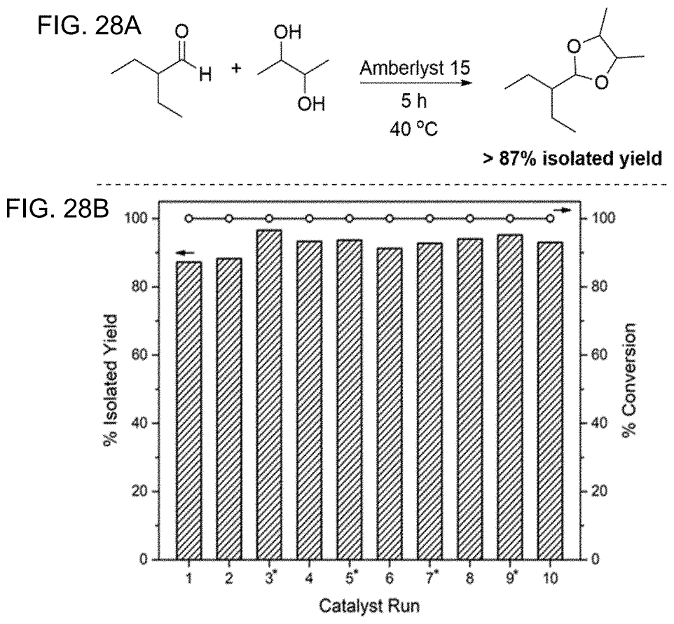

The catalyst system was investigated, bearing in mind the requirement of a slight BDO excess and generation of water as a side-product. In a similar reaction system, it was reported²³ that during acetone condensation, Amberlyst CH28 was deactivated due to water build up on the catalyst surface. Therefore, the stability of Amberlyst 15 was investigated for the acetalization of compounds 1-5. To test the catalyst recyclability, the reaction of 2-ethylbutyraldehyde with BDO was selected to prepare (1) (FIG. 28A). 2-Ethylbutyraldehyde (3.215 g, 32.10 mmol), 2,3-Butanediol (4.963 g, 55.07 mmol), and Amberlyst 15 (0.350 g) were combined in a 20 mL scintillation vial. The apparatus was heated to 40 C with stirring (500 rpm). After 5 h the reaction was complete and allowed to cool for 15 min. Upon cooling, the organic layer was decanted to yield the desired product, 1, in 100% selectivity as evidenced by GCMS. This process was repeated for catalyst runs 2 through 10. To ensure efficient mixing between the aldehyde, BDO, and catalyst, the aqueous layer was removed upon completion of runs 3, 5, 7, and 9. Each successive reaction was loaded with 2-Ethylbutyraldehyde and 2,3-Butanediol, detailed in Table 1. After 10 catalyst recycle runs, the reaction still produced >87% isolated yield of 1, with 100% conversion observed for each of ten catalyst recycle runs, FIG. 28B.

TABLE 1
Catalyst Recycle Results

| | 2-Ethylbutyraldehyde | 2,3- | Isolated |
|---|---|---|---|
| Run | (mmol) | Butanediol (mmol) | yield (%) |
| 1 | 32.10 | 55.07 | 87.2 |
| 2 | 32.05 | 53.99 | 88.2 |
| 3 | 31.30 | 55.84 | 96.6 |
| 4 | 31.66 | 56.71 | 93.3 |
| 5 | 31.96 | 60.25 | 93.7 |
| 6 | 32.51 | 59.55 | 91.2 |
| 7 | 32.03 | 55.69 | 92.7 |
| 8 | 31.99 | 57.45 | 94.0 |
| 9 | 31.95 | 58.08 | 95.2 |
| 10 | 32.07 | 56.92 | 93.0 |

Without wishing to be bound by theory, it is understood that the solubility of water in BDO likely prevented competitive inhibition on the catalyst surface and afforded the high degree of catalyst recyclability, as evidenced above. It is worthwhile to note that the conversion for each of the ten catalyst recycle runs was 100% and the discrepancy between the conversion and isolated yield was due to the workup procedure. The product was decanted away from the aqueous phase, and the ability to remove all of the product was limited due to the desire to eliminate any use of an organic solvent for extraction purposes. While the isolated yields were not unity, the absence of additional solvents or purification steps demonstrate the simplicity and sustainability of this method. This property, as well as the employment of a solid acid catalyst, could allow this method to easily be translated to a continuous flow reactor, for large scale production.

### Example 9: Analysis of Fuel Properties

The physical properties of the five distinct dioxolanes obtained were analyzed to understand their potential as suitable fuel components.^{4-5, 8, 16, 18-20} A tabulated summary of these properties are provided in Table 2. The first metric considered was viscosity. While many biodiesels²⁴ are significantly more viscous than their petroleum counterparts²⁵, compounds 1-5 exhibited kinematic viscosities significantly lower than biodiesel, H1-H3, and petroleum diesel at temperatures ranging from −20 to 40° C. If a fuel's viscosity falls outside of the ASTM D 975 specification (>4 mm² s⁻¹), it often leads to poor atomization of the fuel, which results in poor combustion and a subsequent loss of power and decrease in fuel economy.⁷ In this respect, compounds 1-5 could be used to thin biodiesel to meet standards for petroleum diesel.

In addition to high viscosities, many biodiesels have high freezing points²⁴, which lie at temperatures near or even above the freezing point of water, which is another drawback to their extensive application. Compounds 1-5 exhibited freezing points below −100° C. Without wishing to be bound by theory, it is understood that the low freezing point of compounds 1-5 was due to each of the compounds existing as a mixture of three diastereomers. These fuels, with their low freezing points, offer a significant improvement over biodiesel, H1, H2, H3, and petroleum diesel. Additionally, compounds 1-5 had molecular weights between 49 and 68% that of soy biodiesel,²⁴ for the C₈ and C₁₂ acetals, respectively. It is understood that their low molecular weight contributed to the low viscosity and freezing point observed for compounds 1-5.

Two additional factors to consider for diesel fuel formulation are density and net heat of combustion (NHOC). Typically, for a chemical compound to be able to be blended with diesel, it should not decrease the density of the fuel mixture below 0.82 g mL-¹, as the energy content of the fuel brought into the engine is dependent on the density of the fuel.^{17, 25} Compounds 1-5 maintained densities slightly higher than that of biodiesel, H1-H3, and petroleum diesel. A related metric is the NHOC, which is a measure of the energy contained in a given quantity of a compound. Additionally reported in Table 2 is the lower heating value (LHV) of compounds 1-5. The LHV of a fuel is defined as the amount of heat released by combusting a specified quantity (initially at 25° C.) and returning the temperature of the combustion products to 150° C., which assumes the latent heat of vaporization of water in the reaction products is not recovered.

Petroleum diesel is prone to producing a high level of soot during combustion.²⁶ One method of determining how cleanly a fuel burns is via determination of the yield sooting index (YSI), which provides a measure of soot formation rates.²⁷⁻²⁸ When compared to petroleum diesel, compounds 1-5 displayed much lower YSI values, and therefore would likely burn cleaner.

Another important measure of the quality of a diesel fuel is the cetane number (CN), which is a dimensionless measurement typically ranging from 0-100 (though measurements outside this range are possible), and measures how readily a fuel autoignites in a compression ignition engine. Using ignition quality testing (IQT), the derived cetane number (DCN, ASTM D689D) was obtained, which is a reliable predictor of CN. It was observed that only compound 3 would not meet diesel standards (a minimum cetane number of about 40 is required for most of North America),²⁵ while compounds 1, 2, 4 and 5 exhibited DCN values comparable to, or exceeding that of winter diesel. This suggests that compounds 1-5 (excluding 3) could be readily blended with either petroleum diesel or biodiesel to reduce the viscosity and help to depress the freezing point, without hampering the combustion performance of the fuel. Additionally, the cetane number of the branched acetals was approximately 4 units lower than their corresponding straight chain counterparts. Without wishing to be bound by theory, increasing carbon number is a potentially more efficient way to increase cetane number, which can be seen by comparing compounds 1-5 to dioxolanes H1-H3.

TABLE 2
Fuel properties for compounds 1-5, other dioxolanes, FAME biodiesel, and winter diesel.

| | Density | | Viscosity | Freezing/ | Water | | | | Boiling | Flash |
|---|---|---|---|---|---|---|---|---|---|---|
| | (25° C., | NHOC | (40° C., | MP | Solubility | | | LHV | Point | Point |
| Fuel | g mL⁻¹) | (MJ L⁻¹) | mm² s⁻¹) | (° C.)^{a} | (mg/l) | DCN | YSI | (MJ kg⁻¹) | (° C.) | (° C.) |
| 1 | 0.893 | 29.45 | 1.26 | <−100 | <40 | 44.8 | 58 | 32.98 | 174 | 54.4 |
| 2 | 0.885 | 30.41 | 1.88 | <−100 | <40 | 64.2 | 69 | 34.36 | 184 | 58.3 |
| 3 | 0.894 | 27.67 | 0.94 | <−100 | <40 | 33.4 | 36 | 30.95 | 161 | 42.5 |
| 4 | 0.887 | 29.20 | 1.49 | <−100 | <40 | 48.1 | 49 | 32.92 | 177 | 69.5 |
| 5 | 0.883 | 30.06 | 2.34 | <−100 | <40 | 68.9 | 63 | 34.04 | 188 | 79.5 |
| H1^{b} | 0.868 | 31.99 | 4.44 | −51/−18 | — | 84 | — | — | — | — |
| H2^{b} | 0.872 | 31.99 | 5.15 | −48/−14 | — | 91 | — | — | — | — |
| H3^{b} | 0.883 | 32.28 | 4.98 | −25/0 | — | 81 | — | — | — | — |
| Soy | 0.882 | 32.63 | 4.26 | 0/−4 | — | 51.3* | — | — | — | — |
| bio-diesel^{c} | | | | | | | | | | |
| Palm | 0.873 | 32.55 | 4.61 | 14/13 | — | 61.9* | — | — | — | — |
| bio-diesel^{c} | | | | | | | | | | |
| Winter | 0.843-0.830 | 35.96 | 2.51-2.67 | <−21 | — | 45-54* | 240 | — | — | — |
| diesel^{d} | | | | | | | | | | |

^{a}Freezing and melting points were measured by DSC (Ar = 40 sccm, Tᵣₐₘₚ = 10K min⁻¹, T_{range} = 123-323K).
^{b}Taken from reference 18.
^{c}Taken from reference 23.
^{d}Taken from reference 24, for USA diesel East-West coasts.
*Indicates measured Cetane Number (CN).

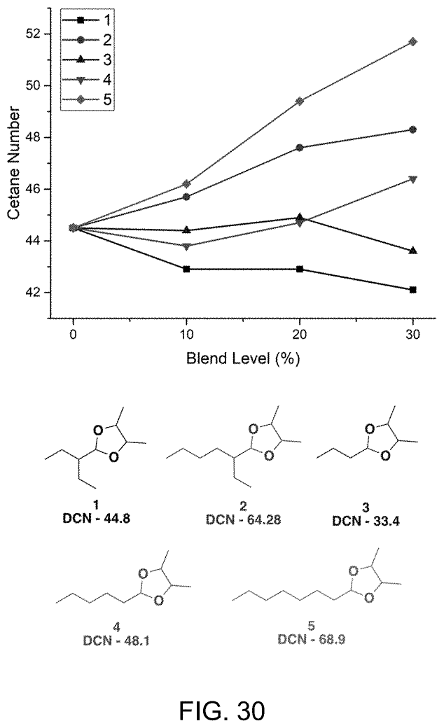

The dioxolanes were blended in percentages of 10%, 20%, or 30% with a 7-component surrogate diesel. The characterization of the 7-component surrogate diesel is provided in Table 3. As seen in FIG. 30, the cetane number generally increased with blends. The data also shows that the components blend linearly.

TABLE 3

| | Molar % | Wt-% | Vol.-% |
|---|---|---|---|
| a-Methylnaphthalene | 12.39 | 9.92 | 8.12 |
| trans-Decalin | 20.08 | 15.61 | 14.99 |
| 2,2,4,4,6,8,8- | 19.00 | 24.20 | 26.11 |
| Heptamethylnonane | | | |
| n-Butylcyclohexane | 10.50 | 8.28 | 8.67 |
| n-Hexadecane | 16.61 | 21.15 | 22.89 |
| Tetralin | 13.78 | 10.25 | 8.83 |
| n-Dodecylbenzene | 7.64 | 10.59 | 10.39 |
| Total | 100.0 | 100.0 | 100.0 |

| | |
|---|---|
| Average Molecular Weight | 177.78 |
| Cetane Number (ICN D8183) | 44.5 |
| Density, g/mL | 0.8430 |
| Cloud point, ° C. | −10.5 |

The cloud point of the blends was also investigated, the results for which are provided in Table 4. The values range from −9.9° C. to −13.3° C. The numbers of the samples correspond with the dioxolanes as-referenced in FIG. 30. Cloud point is a quality specification for diesel fuels. This is an indicator of the tendency of a fuel to form wax crystals when cold. These crystals can clog the atomizer that injects fuel into the combustion chamber of a diesel engine. Cloud point specifications are a maximum temperature. A material with lower cloud point is more desirable.

TABLE 4

| | | | | | |
|---|---|---|---|---|---|
| 10% | −10.3° C. | −10.3° C. | −11.5° C. | −10.1° C. | −9.9° C. |
| 20% | −10.8° C. | −10.8° C. | −12° C. | −10.8° C. | −10.8° C. |
| 30% | −10.8° C. | −12.5° C. | −13.3° C. | −10.4° C. | −11.6° C. |

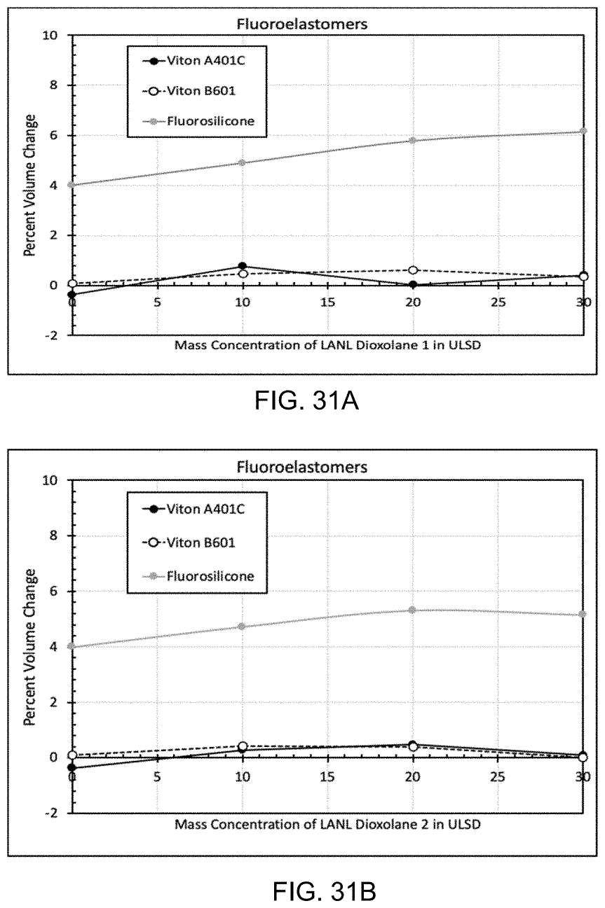

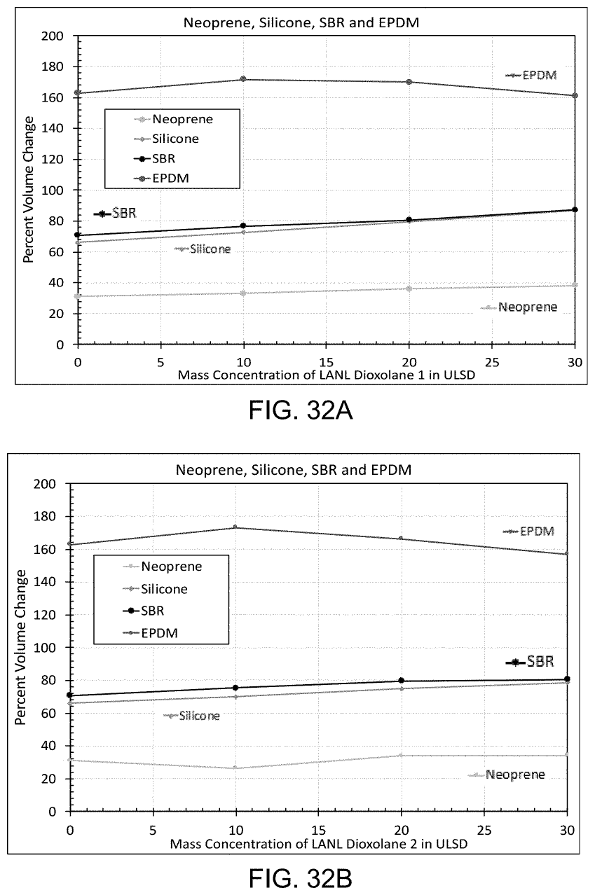

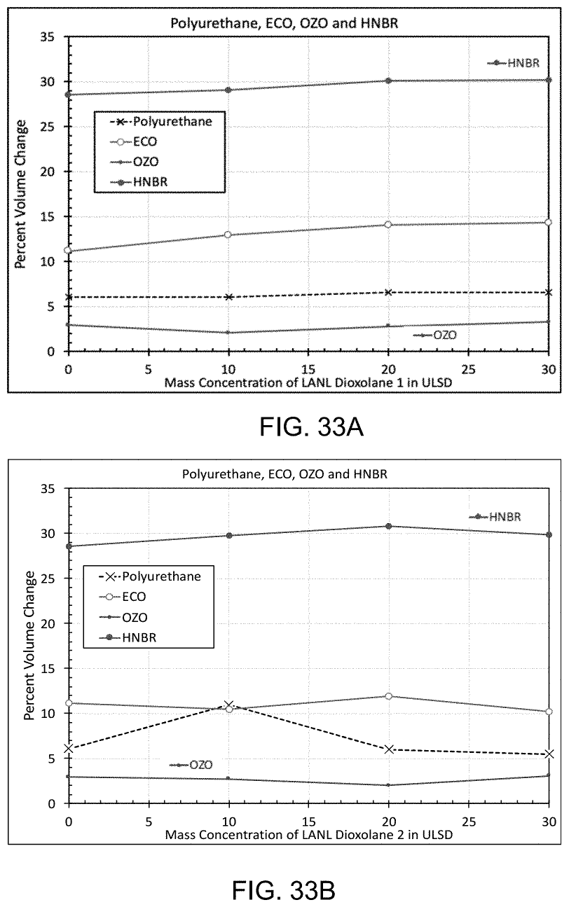

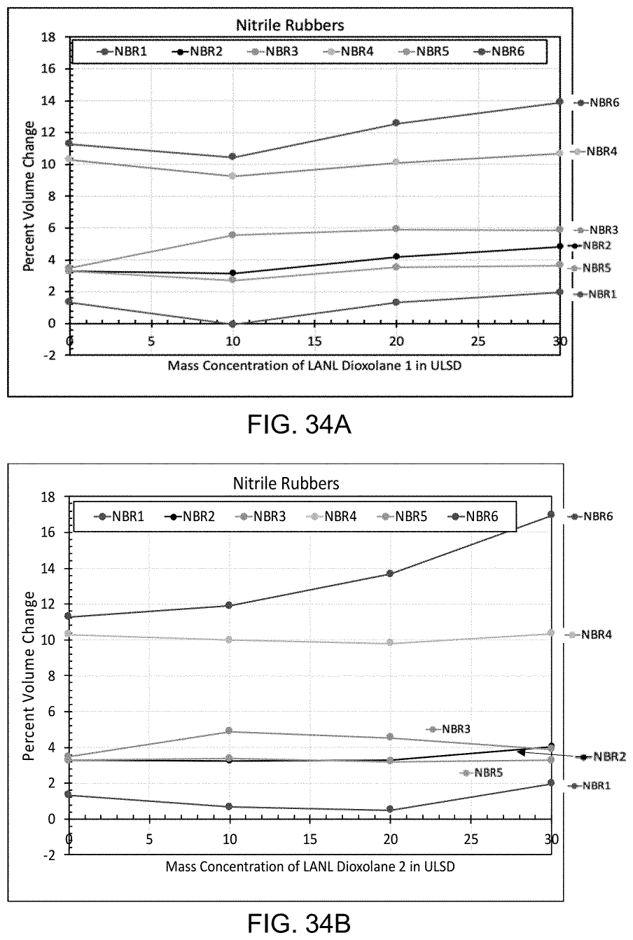

FIGS. 31A, 31B, 32A, 32B, 33A, 33B, 34A, and 34B show the percent volume change of materials that are common components in fuel and engine infrastructure such as fuel pumps and engine seals as a function of mass concentration of dioxolane 1 or 2. Methods were performed in accordance with Kass, Michael, et al. Performance of Vehicle Fuel System Elastomers and Plastics with Test Fuels Representing Gasoline Blended with 10% Ethanol (E10) and 16% Isobutanol (iBu16). United States: N. p., 2020. Materials investigated include viton, flouorsilicones, neoprene, silicone, styrene-butadiene rubber (SBR), Ethylene Propylene Diene Monomer (EPDM) rubber, polyurethane, epichlorohydrin rubber (ECO), Paracril OZO plastic materials, hydrogenated acrylonitrile butadiene rubber (HNBR) and a range of nitrile rubbers. As indicated in the figures, the dioxolanes do not cause adverse volume change in the materials, which means they are compatible with engine infrastructure.

### REFERENCES

1 G. W. Huber, S. Iborra and A. Corma, Chem. Rev., 2006, 106 (9), 4044.
2 P. Fairley, Nature, 2011, 474, S2.
3 R. Henriques, Angew. Chem., 1898, 11 (30), 697.
4 R. L. McCormick, A. W., J. Ireland, M. Brimhall and R. R. Hayes, Effects of biodiesel Blends on Vehicle Emissions, Milestone Report NREL/MP-540-40554. National Renewable Energy Laboratory, 2006.
5 B. S. Sazzad, M. A. Fazal, A. S. M. A. Haseeb and H. H. Masjuki, RSC Adv. 2016, 6 (65), 60244.
6 R. W. Jenkins, C. M. Moore, T. A. Semelsberger, C. J. Chuck, J. C. Gordon and A. D. Sutton, ChemSusChem, 2016, 9 (9), 922-923.
7 D. Singh, K. A. Subramanian, M. Juneja, K. Singh, S. Singh, R. Badola and N. Singh, Prog. Sustainable Energy, 2017, 36 (1), 214.
8 G. Knothe and K. R. Steidley, Fuel, 2005, 84 (9), 1059.
9 Building Partnerships|Growing Markets 2017 Ethanol Industry Outlook: Renewable Fuels Association. http://www.ethanolrfa.org/wpcontent/uploads/2017/02/Ethanol-Industry-Outlook-2017.pdf, accessed April 2018.
10 C. M. Moore, O. Staples, R. W. Jenkins, T. J. Brooks, T. A. Semelsberger and A. D. Sutton, Green Chem., 2017, 19 (1), 169.
11 W. Bertleff, M. Roeper and X. Sava, in Ullmann's Encyclopedia of Industrial Chemistry, Wiley-VCH Verlag GmbH & Co. KgaA, 2007.
12 A. W. Franz, H. Kronemayer, D. Pfeiffer, R. D. Pilz, G. Reuss, W. Disteldorf, A. O. Gamer and A. Hilt, in Ullmann's Encyclopedia of Industrial Chemistry, Wiley-VCH Verlag GmbH & Co. KgaA, 2016.
13 K. K. Ramasamy and Y. Wang, Catal. Today, 2014, 237, 89.
14 M. Schlaf, Dalton Trans., 2006, 39, 4645.
15 Please refer to reference 10 for discussion of fuel properties of alkanes derived from HDO
of aldehydes produced from acetaldehyde.
16 K. Górski and M. Przedlacki, Energy Fuels, 2014, 28 (4), 2608.
17 Chevron Corporation. Diesel Fuels Technical Review; 2007.
18 K. W. Harrison and B. G. Harvey, Sustainable Energy Fuels, 2018, 2 (2), 367.
19 H. Liu, X. Bi, M. Huo, C.-f. F. Lee and M. Yao, Energy Fuels, 2012, 26 (3), 1900.
20 G. Sørensen, D. V. Pedersen, A. K. Nørgaard, K. B. Sorensen and S. D. Nygaard, Bioresour. Technol., 2011, 102 (8), 5259.
21 S. Yang, A. Mohagheghi, M. A. Franden, Y.-C. Chou, X. Chen, N. Dowe, M. E. Himmel and M. Zhang, Biotechnol. Biofuels, 2016, 9 (1), 189.
22 B. G. Harvey, W. W. Merriman, R. L. Quintana ChemSusChem, 2016, 9, 1814.
23 S. Talwalkar and S. Mahajani, Appl. Catal., A, 2006, 302 (1), 140.
24 S. K. Hoekman, Renewable Sustainable Energy Rev., 2012 16 (1), 143.
25 Infinium International, Ltd. Worldwide Winter Diesel fuel Quality Survey 2016; Infineum International, Ltd., Abingdon, U. K., 2016.
26 D. D. Das, C. S. McEnally, T. A. Kwan, J. B. Zimmerman, J. W. Cannella, C. J. Mueller, L. D. and Pfefferle, Fuel, 2017, 197, 445.
27 D. D. Das, P. C. St. John, C. S. McEnally, S. Kim and L. D. Pfefferle, Combust. and Flame 2018, 190, 349.
28 C. S. McEnally, Y. Xuan, P. C. St. John, D. D. Das, A. Jain, S. Kim, T. A. Kwan, L. K. Tan, J. Zhu and L. D. Pfefferle, Proc. Combust. Inst., 2018, in press.
29 Y. Li, Y. Wu, J. Zhu, J. Liu, Y. Shen, J. Saudi Chem. Soc. 2016, 20, S495.

Efforts have been made to ensure accuracy with respect to numbers used (e.g. amounts, temperature, etc.) but some experimental errors and deviations should be accounted for.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The disclosures of all cited references including publications, patents, and patent applications are expressly incorporated herein by reference in their entirety.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller ranges is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. One skilled in the art will recognize many methods and materials similar or equivalent to those described herein, which could be used in the practicing the subject matter described herein. The present disclosure is in no way limited to just the methods and materials described.

What is claimed is:

1. A process for the preparation of a diesel fuel additive compound of Formula I, 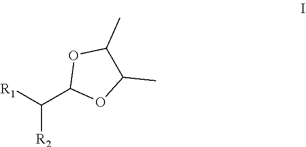
wherein R₁ is C₁₋₈ alkyl or C₂₋₆ alkyl and R₂ is selected from the group consisting of H, C₁₋₈ alkyl, and C₂₋₆ alkyl,
the process comprising: 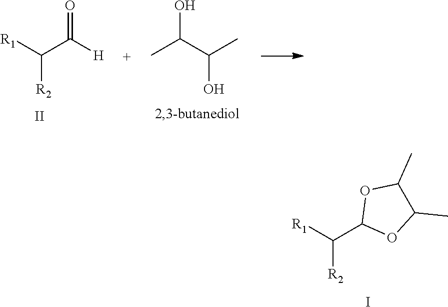
contacting a compound of formula II with 2,3-butanediol in the presence of a catalyst to form a solution;
heating said solution at a temperature of about 40° C.; and
allowing said solution to separate into an upper phase and a lower phase.

2. The process of claim 1, further comprising separating the upper phase from said solution, wherein the upper phase comprises the compound of Formula I.

3. The process of claim 2, further comprising repeating said contacting, wherein said 2,3-butanediol is unreacted 2,3-butanediol from the lower phase.

4. The process of claim 1, wherein the catalyst is NAFION.

5. The process of claim 1, wherein the 2,3-butanediol is present in an amount ranging from about 1.25 to about 2.75 moles relative to 1 mole of the compound of Formula II.

6. The process of claim 1, wherein, R¹ is selected from the group consisting of C₂ alkyl, C₄ alkyl, and C₆ alkyl; and R² is C₂ alkyl or H.

7. The process of claim 1, wherein said diesel fuel additive compound of Formula I is selected from the group consisting of

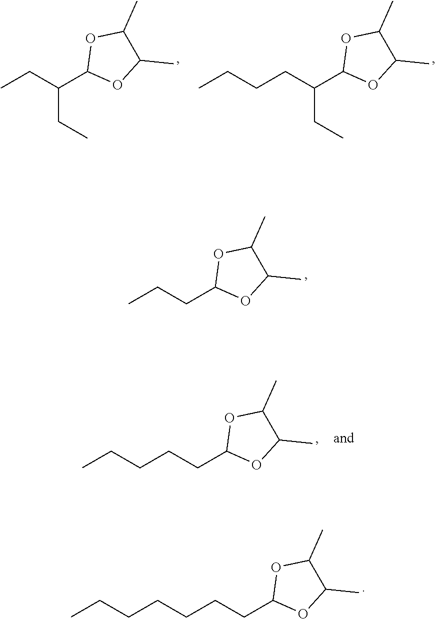

8. A method of improving a property of a diesel fuel, comprising:
contacting a diesel fuel with a compound of Formula I, 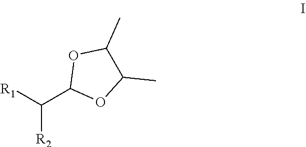
wherein R₁ is C₁₋₈ alkyl or C₂₋₆ alkyl and R₂ is selected from the group consisting of H, C₁₋₈ alkyl, and C₂₋₆ alkyl,
wherein said property is selected from the group consisting of cetane number, freezing point, viscosity, yield sooting index, net heat of combustion, density, and cloud point.

9. The method of claim 8, wherein said property is viscosity and said compound of Formula 1 has a viscosity of about 0.9 mm² s⁻¹ to about 2.4 mm² s⁻¹ at 40° C.

10. The method of claim 8, wherein said property is cetane number and said compound of Formula 1 has a derived cetane number of about 45 to about 70.

11. The method of claim 8, wherein said property is freezing point and said compound of Formula I has a freezing point of less than −100° C.

12. The method of claim 8, wherein said property is density and said compound of Formula I has a density in a range of about 0.880 g/mL to about 0.895 g/mL at 25° C.

13. The method of claim 8, wherein said property is net heat of combustion and said compound of Formula I exhibits a net heat of combustion of about 27.60 to about 30.50 MJ/L.

14. The method of claim 8, wherein said property is yield sooting index, wherein said compound of Formula I exhibits a yield sooting index of about 35 to about 70.

15. The method of claim 8, wherein said property is cetane number, and said compound of Formula I improves said cetane number of said diesel fuel by at least 3%.

16. The method of claim 8, wherein said property is cloud point, and said compound of Formula I improves said cloud point of said diesel fuel by at least 2%.

17. The method of claim 8, wherein R¹ is selected from the group consisting of C₂ alkyl, C₄ alkyl, and C₆ alkyl; and R² is C₂ alkyl or H.

18. A diesel fuel composition comprising a diesel fuel and a compound of Formula I, 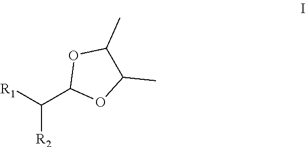
wherein R₁ is C₁₋₈ alkyl or C₂₋₆ alkyl and R₂ is selected from the group consisting of H, C₁₋₈ alkyl, and C₂₋₆ alkyl.

19. The diesel fuel composition of claim 18, wherein said compound is mixed with said diesel fuel in approximately a 1:10 to 5:10 ratio.

20. The diesel fuel composition of claim 18, wherein, R¹ is selected from the group consisting of C₂ alkyl, C₄ alkyl, and C₆ alkyl; and R² is C₂ alkyl or H.
